(19) United States
(12) Patent Application Publication
(10) Pub. No.: US 2025/0235413 A1
(43) Pub. Date: Jul. 24, 2025

(54) MODIFIED FORMS OF AMBROXOL FOR THERAPEUTIC USE

(71) Applicant: Zywie, LLC, Princeton, NJ (US)

(72) Inventors: Stephen Anderson, Princeton, NJ (US); Stephen Pasternak, London (CA); Vincent Jacques, Somerville, MA (US); Gerard Joseph Broussard, JR., Langhorne, PA (US)

(73) Assignee: Zywie, LLC, Princeton, NJ (US)

(21) Appl. No.: 18/705,344

(22) PCT Filed: Oct. 27, 2022
(86) PCT No.: PCT/US2022/078779

§ 371 Date: Apr. 26, 2024

Related U.S. Application Data
(60) Provisional application No. 63/272,743, filed on Oct. 28, 2021.

(51) Int. Cl. A61K 31/137; A61K 9/00; A61K 9/16; A61P 25/28; C07C 215/70

(52) U.S. Cl. CPC A61K 31/137; A61K 9/0056; A61K 9/1652; A61P 25/28; C07C 215/70; C07B 2200/05

(57) ABSTRACT

The invention relates to polydeuterated analog forms of ambroxol and related compounds (including bromhexine and the ambroxol salt, ambroxol hydrochloride), compositions comprising same, and methods of preventing and/or treating various diseases and medical conditions involving the administration of polydeuterated analogs of ambroxol and related compounds.

### BACKGROUND OF THE INVENTION

### Field of the Invention

The invention relates to modified forms of ambroxol, ambroxol hydrochloride, and/or bromhexine suitable for use in various therapeutic applications.

### Discussion of the Related Art

Ambroxol, ambroxol hydrochloride and the structurally related “parent compound” bromhexine, are mucolytic agents that have been available in a number of countries since the late 1970s for use in the treatment of acute and chronic respiratory diseases and conditions associated with the production of excess and/or highly viscous mucus.

By triggering lysosomal exocytosis via pH-dependent calcium release from acidic calcium stores (Fois G et al., Cell Calcium 58 (6): 628-637, 2015), it is thought that ambroxol acts to promote mucus clearance by, for example, breaking up phlegm, and stimulating the production of surfactant by type II pneumocytes (Seiffert C et al., Toxicol Appl Pharmacol 203 (1): 27-35, 2005) to reduce adhesion of mucus to the walls of the respiratory tract. In addition, it has been found that ambroxol potently inhibits neuronal sodium channels to enable, especially when administered in the form of a lozenge, rapid pain relief used in acute sore throat (de Mey C et al., Arzneimittel-Forschung 28 (5a): 889-898, 1978).

In more recent times, there has been wide interest in the repurposing of ambroxol for a number of other medical uses. For example, it has been reported that ambroxol can act as a “molecular chaperone” for the lysosomal enzyme beta-glucocerebrosidase (GCase, UniProtKB Entry P04062) to thereby increase the activity of this enzyme. This may mean that ambroxol might be suitable for the treatment of Gaucher's disease (Maegawa G H B et al., J Biol Chem 284 (35): 23502-23516, 2009), which is the most prevalent lysosomal storage disease, and is caused by a deficiency in GCase. Similarly, this ability to increase the activity of GCase may be beneficial for the treatment of Parkinson's disease (PD) in individuals with loss-of-function mutations in the glucocerebrosidase gene, GBA1, (McNeill A et al., Brain 137 (5): 1481-1495, 2014). In a recent report, it has been shown that daily ambroxol administration was able to increase brain GCase activity in healthy, non-human primates (Migdalska-Richards A et al., Synapse 71 (7): €21967, 2017).

Accordingly, ambroxol continues to be the subject of considerable research interest and effort. The present inventors, in the course of their work towards identifying and developing new therapeutic methods and compositions based upon ambroxol, have designed novel modified forms of ambroxol (i.e., ambroxol analogs). It is considered that these modified compounds may offer one or more advantage over one or more of ambroxol, ambroxol hydrochloride and bromhexine such as, for example, greater stability leading to an increased half-life and duration of action in the body.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The invention relates to polydeuterated analog forms of ambroxol and related compounds (including bromhexine, its pharmaceutically acceptable salts, and pharmaceutically acceptable salts of ambroxol such as ambroxol hydrochloride), compositions comprising same, and methods of preventing and/or treating various diseases and medical conditions involving the administration of polydeuterated analogs of ambroxol and related compounds.

More specifically, the invention provides, particularly, a polydeuterated compound according to formula I:

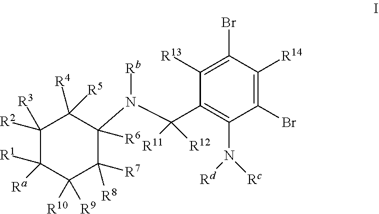

wherein
R^{a} is selected from H, hydroxyl (OH), lower alkyl (e.g., C₁₋₃ alkyl such as CH₃ and CH₂CH₃), and lower alcohol (e.g., C₁₋₃ alcohol such as CH₂—OH) wherein optionally one or more H atoms in any of the aforementioned groups is replaced by deuterium (D),
R^{b} is selected from H, deuterium (D) and

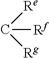

where R^{e}, R^{f} and R^{g} are independently selected from H and D,
R^{c} and R^{d} are independently selected from H and D, and
each of R¹ to R¹⁴ are independently selected from H and D;
wherein the polydeuterated compound comprises at least two deuterium (D) atoms, and with the proviso that the compound is not bis-deuterated ambroxol A or [D₁₁]-ambroxol B shown below;
or a pharmaceutically acceptable salt, solvate or prodrug thereof.

### Bis-Deuterated Ambroxol A (ZW-001):

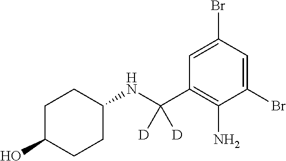 [SMILES: [2H]C([2H])(N[C@H]1CC[C@H](O)CC1)c1cc(Br)cc(Br)c1N]

### [D₁₁]-Ambroxol B:

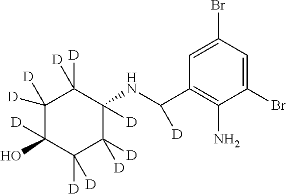 [SMILES: [2H]C(N[C@]1([2H])C([2H])([2H])C([2H])([2H])[C@@]([2H])(O)C([2H])([2H])C1([2H])[2H])c1cc(Br)cc(Br)c1N]

It is contemplated that any or all H atoms in the compound of formula I may be replaced by a deuterium atom. In some embodiments, the compound comprises 2, 3, 4, 5, 6, 7, 8, 9, 10, 11, 12, 13, 14, 15, 16, 17, 18, 19 or 20 deuterium (D) atoms. Preferably, the compound comprises at least 3 deuterium (D) atoms.

In some embodiments, the deuterium atoms are provided only on the ring structures of the compound of formula I. For example, the dibromophenylaniline ring may comprise up to 2 deuterium (D) atoms and the optionally substituted cyclohexyl ring may comprise up to 10 deuterium (D) atoms. Optionally, the amine substituent present on the dibromophenylaniline ring may comprise 1 or 2 deuterium (D) atoms; that is, at least one, and preferably both, of R^{c} and R^{d} are D.

In some other embodiments, the deuterium atoms are provided on one or both of the ring structures of compound of formula I, as well as on the linking group between those ring structures. Where deuterated, the linking group may comprise 1 or 2 deuterium (D) atoms.

In some embodiments, at least one, and preferably both, of R¹¹ and R¹² is/are deuterium (D).

In some embodiments, one effect of the polydeuteration of the compound is resistance to metabolism (as compared to the corresponding compound lacking deuteration) such as, for example, metabolism through cleavage (e.g., via oxidation) of the covalent carbon-nitrogen bond of the linking group between the ring structures of compounds according to formula I.

In some specific embodiments, the compound is deuterated: only at each of R¹ to R¹⁰; only at each of R¹ to R¹⁰, R¹¹ and R¹²; only at each of R¹ to R¹⁰, R¹³ and R¹⁴; or only at each of R¹¹ to R¹⁴; while in other specific embodiments, the compound is deuterated at each of R¹ to R¹⁴.

In some embodiments, the compound is a polydeuterated analog of ambroxol or a polydeuterated analog of bromhexine.

In some embodiments, the compound is a pharmaceutically acceptable salt of a polydeuterated analog of ambroxol or bromhexine, preferably a hydrochloride.

The invention also provides a pharmaceutical composition comprising a compound of formula I (or a pharmaceutically acceptable salt, solvate or prodrug thereof), and methods of preventing and/or treating various diseases and medical conditions in a subject involving the administration of a compound of formula I (or a pharmaceutically acceptable salt, solvate or prodrug thereof).

More specifically, the invention provides methods of preventing and/or treating a disease or medical condition selected from the group consisting of respiratory diseases and conditions (e.g., bronchopulmonary diseases, and especially those associated with the production of excess and/or highly viscous mucus) and including pain associated with acute sore throat, lysosomal storage disorders (LSDs) such as Gaucher's disease, neurological diseases and conditions (e.g., PD and other aging-associated diseases) involving dysfunction of autophagy (Dockrill P., ScienceAlert, February 2020).

Additionally, the invention provides methods of treating, reducing and/or stabilizing the symptoms associated with neurological diseases and conditions such as Parkinsonism (including PD and Dementia with Lewy Bodies), Alzheimer's Disease (AD), and/or Pick's disease.

Further, the invention provides methods of extending life expectancy of a subject. Specifically, compounds of formula I may be used in a method of (a) treating, inhibiting, or reducing aging of a subject, (b) treating, inhibiting, or reducing an age-related symptom or an age-related disease in a subject, and/or (c) increasing the healthspan, lifespan, and/or mental acuity of a subject.

In preferred embodiments, the subject is a mammal, and in even further preferred embodiments, the mammal is a human, a domesticated animal (e.g., a dog, a cat, a horse) or a farm animal (e.g., a cow or a pig).

### BRIEF DESCRIPTION OF THE FIGURES

The objects and features of the invention can be better understood with reference to the following detailed description and accompanying figures.

FIG. 1 provides the structures of representative compounds (compounds 1 to 14) of the invention.

FIG. 2 provides the structures of ZW-001 (1) (bis-deuterated ambroxol A), ZW-002 (2) (also referred to herein as “Compound 1”), ZW-003 (3) (also referred to herein as “Compound 3”) and ambroxol (4).

FIG. 3 provides animal data showing that ambroxol was able to increase lifespan in a mouse animal model. Group 1 represents control animals that were not administered ambroxol; and Group 2 represents animals administered 50 mg/kg (body weight) of ambroxol daily as a chow supplement, starting at 2 months of age.

FIG. 4 shows results obtained from mice treated with ambroxol at age 7 months and tested for novel place recognition. This is a cognitive test to measure short-term working memory, involving recognition of a familiar object found in an unfamiliar place (see Magen et al., Eur J Neurosci 35:870-882, 2012; Magen and Chesselet, J Parkinson's Disease 1:217-227, 2011). A Discrimination Index (DI)=(tₙₒᵥₑₗ−t_{familiar})/(tₙₒᵥₑₗ+t_{familiar}) was used to assess the time spent exploring near the object in a novel place (“tₙₒᵥₑₗ”) vs. the total exploration time overall. Discrimination Index scores greater than zero are considered to indicate good place recognition memory.

FIG. 5 shows the effect of ambroxol on basal macroautophagy in mouse cells. Mouse fibroblasts in culture (NIH3T3 cells) expressing the tandem reporter mCherry-GFP-LC3 were exposed to the indicated concentrations of ambroxol for 24 h in complete media. Panel A. Schematic of the autophagic compartments analyzed. Panels B-D. Number of autophagic vacuoles (AV) (Panel B); autophagosomes (APG) (Panel C); and autolysosomes (AUT) (Panel D). All values are mean+s.e.m. and quantifications were done in at least 2,500 cells per condition in three different experiments using high content microscopy. Differences with untreated (0 μM ambroxol) are significant for *p<0.05 **p<0.01 and ***p<0.001.

FIG. 6 shows the effect of deuterated ambroxol on human iPSC-derived neurons. In the figure, the compound designated ZW-001 is the compound otherwise described herein as bis-deuterated ambroxol A, and the compound designated ZW-002 in the figure is the compound otherwise referred to herein as compound 1.

FIG. 7 shows the effect of deuterated ambroxol on lysosomal, autophagosomal and TFEB gene expression. In the figure, the compound designated ZW-001 is the compound otherwise described herein as bis-deuterated ambroxol A, and the compound designated ZW-002 in the figure is the compound otherwise referred to herein as compound 1.

### DETAILED DESCRIPTION

### I. Definitions

The following definitions are provided for specific terms which are used in the following written description.

As used in the specification and claims, the singular form “a”, “an” and “the”, include plural references unless the context clearly dictates otherwise.

The present invention can “comprise” (open ended) or “consist essentially of” the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention.

As used herein, a “subject” is a vertebrate, preferably a mammal, more preferably a human, and even more preferably a domesticated animal, such as a pet, or a farm animal. Mammals include, but are not limited to, murines, simians, humans, farm animals, sport animals, and pets. In other preferred embodiments, the “subject” is a rodent (e.g., a guinea pig, a hamster, a rat, a mouse), murine (e.g., a mouse), canine (e.g., a dog), feline (e.g., a cat), equine (e.g., a horse), a primate, simian (e.g., a monkey or ape), a monkey (e.g., marmoset, baboon), or an ape (e.g., gorilla, chimpanzee, orangutan, gibbon). In other embodiments, non-human mammals, especially mammals that are conventionally used as models for demonstrating therapeutic efficacy in humans (e.g., murine, primate, porcine, canine, or rabbit animals) may be employed. In preferred embodiments, an “individual” or “patient” (as in the subject of the treatment) means mammals, particularly non-human primates, e.g., apes and monkeys, and most particularly humans.

As understood herein, an “effective amount” of a pharmaceutical composition of the present invention refers to an amount of the composition suitable to elicit a therapeutically beneficial response in the subject, e.g., promoting mucus clearance associated with respiratory diseases and conditions, providing relief from pain associated with acute sore throat, amelioration of symptoms associated with lysosomal storage disorders (LSDs) and neurological diseases and conditions, or extending and/or increasing and/or improving healthspan, lifespan and/or mental acuity, such as for example, increasing survival and/or healthy aging and/or decreasing morbidity or age-related illness in the subject.

The term “dose” or “dosage” as used herein refers to physically discrete units suitable for administration to a subject, each dosage containing a predetermined quantity of the active pharmaceutical ingredient calculated to produce a desired response.

The term “about” or “approximately” means within an acceptable range for the particular value as determined by those skilled in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term “about” means within an acceptable error range for the particular value, such as ±1-20%, preferably ±1-10% and more preferably ±1-5%. In even further embodiments, “about” should be understood to mean +/−5%.

Where a range of values is provided, it is understood that each intervening value, between the upper and lower limits of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All percentages and ratios used herein are by weight of the total composition unless otherwise indicated herein. All temperatures are in degrees Celsius unless specified otherwise. All measurements made are at 25° C. and normal pressure unless otherwise designated.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by those skilled in the art. This includes, at very least, the degree of expected experimental error, technique error and instrument error for a given technique used to measure a value.

Where used herein, the term “and/or” when used in a list of two or more items means that any one of the listed characteristics can be present, or any combination of two or more of the listed characteristics can be present. For example, if a composition of the present invention is described as containing characteristics A, B, and/or C, the composition can contain A feature alone; B alone; C alone; A and B in combination; A and C in combination; B and C in combination; or A, B, and C in combination.

As used herein, the term “lower alkyl” includes straight chain alkyl groups, branched alkyl groups and cyclic alkyl groups having from 1 to 8 carbon atoms (e.g., methyl, ethyl, propyl, isopropyl, butyl, isobutyl, tert-butyl, pentyl, hexyl, cyclopropyl, cyclobutyl, cyclopentyl, etc.).

As used herein, the term “lower alcohol” includes alcohol groups comprising straight chain, branched or cyclic alkyl groups having from 1 to 8 carbon atoms and 1 or more hydroxyl (OH) groups (e.g., methanol, ethanol, propanol, etc.).

The term “pharmaceutically acceptable salt” as used herein, refers to salts that retain the desired biological activity of the compound of formula I, and include pharmaceutically acceptable acid addition salts and base addition salts. Suitable pharmaceutically acceptable acid addition salts of the compounds of formula I may be prepared from an inorganic acid or from an organic acid. Examples of such inorganic acids are hydrochloric, sulfuric and phosphoric acid. Appropriate organic acids may be selected from aliphatic, cycloaliphatic, aromatic, heterocyclic, carboxylic and sulfonic classes of organic acids, examples of which are formic, acetic, propionic, succinic, glycolic, gluconic, lactic, malic, tartaric, citric, fumaric, maleic, alkylsulfonic and arylsulfonic. Additional information on pharmaceutically acceptable salts can be found in Remington's Pharmaceutical Sciences, 19th Edition, Mack Publishing Co, Easton PA 1995.

The term “solvate” refers to any form of a compound of formula I, resulting from solvation of with an appropriate solvent. Such a form may be, for example, a crystalline solvate or a complex that maybe formed between the solvent and the dissolved compound.

The term “prodrug” means a compound that undergoes conversion to a compound of formula I within a biological system, usually by metabolic means (e.g., by hydrolysis, reduction or oxidation). For example, an ester prodrug of a compound of formula I containing a hydroxyl group may be convertible by hydrolysis in vivo to the compound of formula I. Suitable esters of the compounds of formula I containing a hydroxyl group may be, for example, acetates, citrates, lactates, tartrates, malonates, oxalates, salicylates, propionates, succinates, fumarates, maleates, methylene-bis-P-hydroxynaphthoates, gestisates, isethionates, di-p-toluoyltartrates, methanesulfonates, ethanesulfonates, benzenesulfonates, p-toluenesulfonates, cyclohexylsulfamates and quinates. As another example, an ester prodrug of a compound of formula I containing a carboxy group may be convertible by hydrolysis in vivo to the compound of formula I. Examples of ester prodrugs include those described by Leinweber F J, Drug Metab Rev 18:379-439 (1987). Similarly, an acyl prodrug of a compound of formula I containing an amino group may be convertible by hydrolysis in vivo to the compound of formula I. Examples of prodrugs for these and other functional groups, including amines, are provided in Prodrugs: challenges and rewards, Valentino J Stella (ed), Springer, 2007.

A “derivative” compound, as the term is used herein, refers to a second compound that is derived from a first compound, such as a brominated version of a non-brominated parent compound. For example, ambroxol is a derivative of the parent compound, bromhexine.

As used herein, the term “deuterium”, “D” and “deuterated” where used to describe the hydrogen atom located at a particular position (e.g., R¹, R² etc.) on a compound of formula I, is to be understood as meaning that in a preparation of the compound, the abundance of deuterium at the particular position is above that which would be naturally expected (i.e., above 0.015%), and would preferably be at least 90% (i.e., in a preparation of the compound, deuterium would be present at the particular position in at least 90% of the molecules comprising the preparation), and more preferably, at least 95% or at least 98%.

As used herein, “lifespan” means the time until death. As used herein “healthspan” or “healthy aging” means the time of life living free (or optimally free) of serious disease. As used herein, “mental acuity” is a measure of a subject's cognitive abilities, such as ability to focus, attention span, and sharpness.

As used herein, “nutrient sensing” is a cell's ability to sense and respond to fluctuations in nutrient levels as is described in Efeyan et al., “Nutrient Sensing Mechanisms and Pathways,” Nature 517:302-310 (2015) (hereby incorporated by reference in its entirety).

As used herein “treating, inhibiting, and/or reducing aging, an age-related symptom, and/or an age-related disease” or the like means reducing the risk of occurrence, delaying the onset, slowing the progression, and/or reducing the severity and/or manifestation, of a sign of aging and/or degenerative disorder, and includes, but is not limited to, preventing the occurrence, development or progression of a sign of aging and/or degenerative disorder.

The term “pharmaceutically acceptable carrier” as used herein means any carrier, diluent or excipient which is compatible with the other ingredients of a composition, and which is not deleterious to the intended subject receiving the composition.

### Compounds of the Invention

The compounds of the invention are polydeuterated analog forms of ambroxol and related compounds (including bromhexine and ambroxol hydrochloride) as defined by formula I. Table 1 shows the structures of ambroxol (also known by its chemical name trans-4-((2-amino-3,5-dibromobenzyl)amino)cyclohexanol), bromhexine (also known by its chemical name 2-amino-3,5-dibromo-N-cyclohexyl-N-methylbenzenemethanamine) and ambroxol hydrochloride.

TABLE 1
Structures of ambroxol, bromhexine and ambroxol hydrochloride

| | |
|---|---|
| Ambroxol | 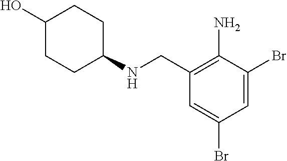 |
| | ambroxol |
| Bromhexine | 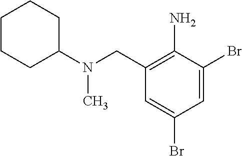 [SMILES: CN(Cc1cc(Br)cc(Br)c1N)C1CCCCC1] |
| | bromhexine |
| Ambroxol Hydrochloride | 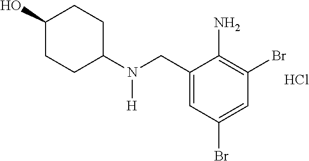 |

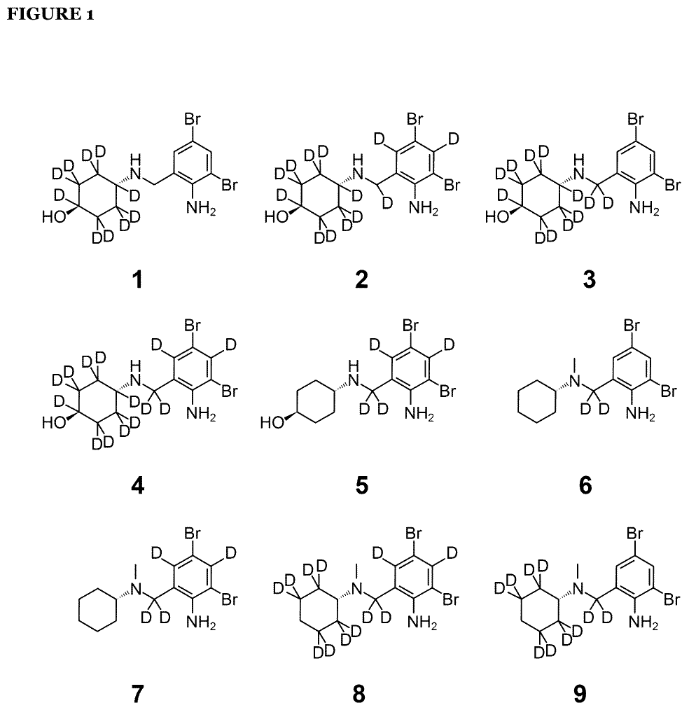

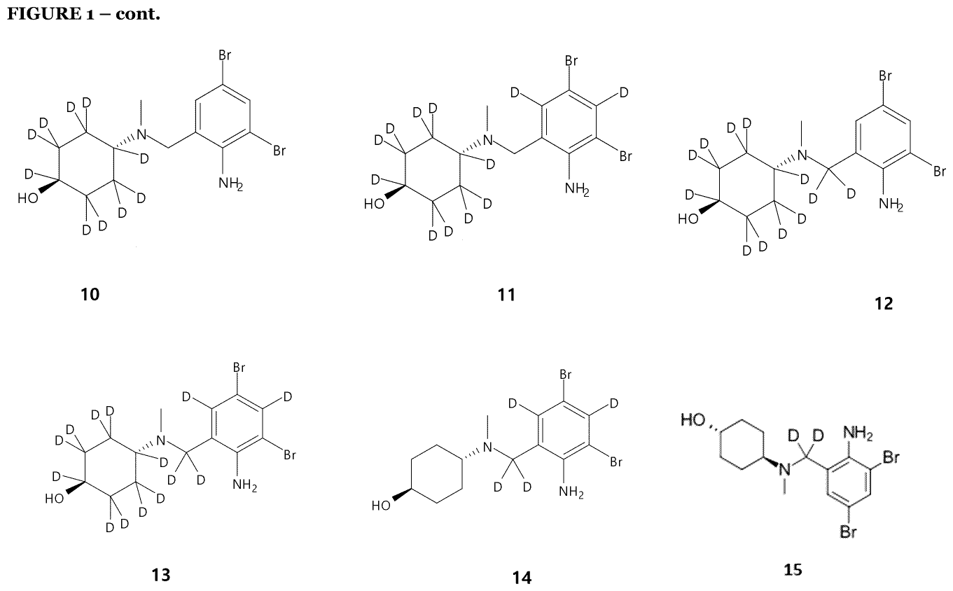

In some embodiments, the compound is selected from the polydeuterated analogs of ambroxol and bromhexine shown in FIG. 1. It is considered that one effect of the polydeuteration of these compounds (particularly compounds 3 to 5) is resistance to metabolism (as compared to the corresponding compound lacking deuteration) such as, for example, metabolism through cleavage (e.g., via oxidation) of the covalent carbon-nitrogen bond of the linking group between the ring structures of compounds according to formula I. In other words, it is considered that one effect of the polydeuteration of these compounds (particularly compounds 3 to 5) is greater stability. Accordingly, in some preferred embodiments, the compound is selected from the polydeuterated ambroxol analog compounds 3 to 5 shown in FIG. 1.

The compounds of the invention may be used in methods of preventing and/or treating various diseases and medical conditions including respiratory diseases and conditions, lysosomal storage disorders (LSDs), and neurological diseases and conditions, and may also be used in methods of extending life expectancy of a subject. In such methods, compounds of the invention showing greater stability (e.g., as compared to a corresponding compound lacking deuteration), may exhibit, for example, one or more advantageous pharmacokinetic properties.

### Preparation of Compounds of the Invention

The compounds of the invention may be prepared by methods known to those skilled in the art of organic synthesis. For example, U.S. Patent Application publication number US 2004/0242700, incorporated herein by reference in its entirety, provides a synthetic protocol for the preparation of ambroxol. This protocol may be readily adapted to enable the synthesis of polydeuterated analog forms of ambroxol and related compounds. In addition, a protocol for the synthesis of ambroxol and related compounds labelled with deuterium (D) is disclosed in Latli B et al., J Label Compd Radiopharm 53:15-23, 2010 (incorporated herein by reference in its entirety), which may also be readily adapted for the preparation of a compound of formula I. For example, “Scheme 4” of Latli et al., 2010 may be adapted for the preparation of a compound of formula I additionally comprising deuterium (D) on the dibromophenylalinine ring by substituting the 2-amino-3,5-dibromobenzaldehyde with a deuterated analog of that compound. Furthermore, compounds of the invention may be prepared by methods such as those exemplified herein.

### Salts of Compounds of the Invention

For compounds that typically contain acidic or basic groups (such as carboxyl or amino groups) such groups will not necessarily be in the free acid or free base form. When referring to compounds of the invention, the reference is intended to include salt forms of the compound. Within the scope of the invention, therefore, are salts of compounds of formula I, especially salts of polydeuterated analogs of ambroxol and bromhexine. The preferred salts are pharmaceutically-acceptable salts.

The term “salts” embraces addition salts of free acids or free bases. The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of therapeutic compounds.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, oxalic, malonic and galacturonic acid. Examples of pharmaceutically unacceptable acid addition salts include, for example, perchlorates and tetrafluoroborates. All of these acid addition salts may be prepared from compounds of formula I by reacting, for example, the appropriate acid with the particular compound.

Suitable pharmaceutically-acceptable base addition salts of compounds of formula I include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methyl glucamine) and procaine. Examples of pharmaceutically unacceptable base addition salts include lithium salts and cyanate salts. All of these base addition salts may be prepared from compounds of formula I by reacting, for example, the appropriate base with the particular compound.

### Methods Using Compounds of the Invention

As mentioned above, the compounds of the invention may be used in methods of preventing and/or treating various diseases and medical conditions including respiratory diseases and conditions, lysosomal storage disorders (LSDs), and neurological diseases and conditions, and may also be used in methods of extending life expectancy of a subject. For example, compounds of formula I may be used in a method of (a) treating, inhibiting, or reducing aging of a subject, (b) treating, inhibiting, or reducing an age-related symptom or an age-related disease in a subject, and/or (c) increasing the healthspan, lifespan, and/or mental acuity of a subject.

In some other embodiments, the invention relates, more specifically, to a method of preventing and/or treating a disease or medical condition in a subject, wherein the disease or condition is selected from the group consisting of respiratory diseases and conditions (e.g., bronchopulmonary diseases, and especially those associated with the production of excess and/or highly viscous mucus) and including pain associated with acute sore throat, lysosomal storage disorders (LSDs) such as Gaucher's disease, and neurological diseases and conditions (e.g., Parkinson's Disease (PD) and other aging-associated diseases involving dysfunction of autophagy), wherein said method comprises administering to the subject an effective amount of a compound of formula I (or a pharmaceutically acceptable salt, solvate or prodrug thereof). The invention also relates to the use of a compound of formula I (or a pharmaceutically acceptable salt, solvate or prodrug thereof) as a medicament for preventing and/or treating a disease or medical condition selected from respiratory diseases and conditions, lysosomal storage disorders (LSDs) and neurological diseases and conditions. In addition, the invention relates to a pharmaceutical composition for a treatment of a disease or medical condition selected from respiratory diseases and conditions, lysosomal storage disorders (LSDs) and neurological diseases and conditions, comprising a therapeutically effective amount of a compound of formula I (or a pharmaceutically acceptable salt, solvate or prodrug thereof), together with a pharmaceutically acceptable carrier.

In some other embodiments, the invention relates to a method of prolonging healthspan, lifespan and/or mental acuity of a subject, comprising administering to the subject an effective amount of a compound of formula I (or a pharmaceutically acceptable salt, solvate or prodrug thereof). The invention also relates to the use of a compound of formula I (or a pharmaceutically acceptable salt, solvate or prodrug thereof) as a medicament for extending life expectancy and/or reducing aging or an age-related illness or symptom. In addition, the invention relates to a pharmaceutical composition for a treatment of extending life expectancy comprising a therapeutically effective amount of a compound of formula I (or a pharmaceutically acceptable salt, solvate or prodrug thereof), together with a pharmaceutically acceptable carrier.

In some further embodiments, the invention relates to a long-term method of inducing increasing and/or improving healthspan, lifespan, and/or mental acuity of a subject, wherein said method comprises administering a therapeutically effective amount of a compound of formula I (or a pharmaceutically acceptable salt, solvate or prodrug thereof), wherein the administration of the compound is at least for 1, 2, 3, 4, 5, 6, 7, 8, 9 or 10 years. With such a method, the lifespan, healthspan, mental acuity and/or healthy aging of the subject is preferably extended, improved, or promoted by up to about 10%, 20%, 30%, 40%, 50%, 60%, or 70% as compared to untreated control subjects.

Examples of age-related illnesses and symptoms include, but are not limited to cardiovascular disease, a metabolic syndrome, a bone-loss disorder, a neurodegenerative disease, pre-diabetes, diabetes, obesity, osteoporosis, coronary artery disease, cerebrovascular disease, heart attack, stroke, peripheral arterial disease, aortic valve disease, stroke, mild cognitive impairment, pre-dementia, dementia, macular degeneration, and cataracts, hair thinning, hair graying, loss of mobility, loss of stamina, fatigue, increased susceptibility to infection, a metabolic change, a biochemical change, cardiac hypertrophy, heart failure, myocardial infarction, ischemia reperfusion injury, inflammatory disease, proinflammatory states, arthropathies, autoimmune diseases, and/or Alzheimer's Disease (AD).

In the methods of the invention, the compound may be administered to the subject in a daily dosage of from about 20-500 mg/day, 50-150 mg/day, 50-200 mg/day, 50-250 mg/day, 250-500 mg/day, or 250 mg-1500 mg/day. Higher or lower doses are also contemplated as it may be necessary to use dosages outside these ranges in some cases. The daily dosage may be divided, such as being divided equally into two to four times per day daily dosing. Moreover, for long-term administration, as might be required in a method of inducing increasing and/or improving healthspan, lifespan, and/or mental acuity of a subject, the compound may, preferably, be administered at a dose of approximately 50 mg/day, 75 mg/day, 100 mg/day, 150 mg/day, 200 mg/day 250 mg/day, 300 mg/day, 350 mg/day, 400 mg/day, 450 mg/day, 500 mg/day, 550 mg/day, 600 mg/day, 650 mg/day, 700 mg/day, 750 mg/day, 800 mg/day, 850 mg/day, 900 mg/day, 950 mg/day, 1000 mg/day, 1050 mg/day, 1100 mg/day, 1150 mg/day, 1200 mg/day, 1250 mg/day, 1300 mg/day, 1350 mg/day, or between 50-150 mg/day, 50-200 mg/day, 50-250 mg/day, 250-500 mg/day, 250 mg-1000 mg/day, or 1000 mg-1500 mg/day or less than 1000 mg/day, or approximately 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, and/or between 4-12 mg/kg/day.

In some preferred embodiments, the methods of the invention may be particularly applied to subjects including humans suffering from pain associated with acute sore throat, human suffering from bronchopulmonary diseases associated with the production of excess and/or highly viscous mucus, humans suffering from a lysosomal storage disorder (LSD) such as Gaucher's Disease, and neurological diseases and conditions such as Parkinsonism (including PD and Dementia with Lewy Bodies), AD, and/or Frontotemporal Dementia.

### Parkinson's Disease and Other Diseases Involving Dysfunctional Autophagy

Ambroxol (and ambroxol hydrochloride and bromhexine) as well as the polydeuterated analogs of ambroxol of the invention may be particularly applied to the treatment of aging-associated diseases involving dysfunction of autophagy and chronic inflammation such as Alzheimer's disease (AD) and Parkinson's disease (PD), and disease states characterized by chronic pain including fibromyalgia.

As mentioned above, the ability of ambroxol to increase the activity of GCase may be beneficial for the treatment of PD in individuals with loss-of-function mutations in the glucocerebrosidase gene, GBA1, (McNeill A et al., 2014 supra), which are prime hereditable risk factors for PD (Do J et al., Neurodegener 14:36, 2019). Enhanced GCase activity resulting from ambroxol treatment is expected to increase the degradative capacity of lysosomes, aiding in the clearance of intracellular aggregated α-synuclein (aSyn), a protein implicated in the pathogenesis of PD (Migdalska-Richards A et al., 2016 supra). GCase deficiency is also associated with lysosomal and mitochondrial dysfunction observed in PD (Brooker and Krainc, 2021 Essays in Biochemistry, 65 (7), pp. 873-883), which may partially explain ambroxol's improvements to lysosome and mitochondrial function (Magalhaes et al., 2018 Scientific Reports, 8 (1), p. 1385). Based on this and promising preclinical data, the use of ambroxol is under investigation in clinical trials as a disease modifying compound for PD (see, for example, Silveira C R A et al., BMC Neurol 19:20, 2019, and Mullin S et al., JAMA Neurol 77:427-434, 2020) and the lipid storage disorder Gaucher's disease (Zimran, Altarescu and Elstein, 2013 Blood Cells, Molecules & Diseases, 50 (2), pp. 134-137; Narita et al., 2016 Annals of Clinical and Translational Neurology, 3 (3), pp. 200-215).

However, the potentially beneficial biological activities of ambroxol are known to extend well beyond its chaperoning of GCase. In particular, ambroxol exerts extensive effects on the autophagic endolysosomal network (AELN) (McNeill et al., 2014 Brain: A Journal of Neurology, 137 (Pt 5), pp. 1481-1495; Fois et al., 2015 Cell Calcium, 58 (6), pp. 628-637; Magalhaes et al., 2018 supra), evoked immune response (Beeh et al., 2008 European Journal of Medical Research, 13 (12), pp. 557-562; Kern and Schwickert, 2017 Journal of Pain Research, 10, pp. 1905-1929), and block of channels involved in chronic pain (Russo et al., 2022 Pain [Preprint]. Available at: https://doi.org/10.1097/j.pain.0000000000002693). As such, ambroxol could potentially prove useful for the treatment of myriad diseases with defects in the AELN, immune system, or nociception as core features. As examples, many neurodegenerative disorders, including PD and AD, exhibit deficits in production or degradation of neurotoxic protein species within the AELN which can in turn drive a chronic inflammatory milieu in brain tissue.

Several of the mechanisms by which ambroxol exerts its impact on cellular processes have been elucidated. Ambroxol is an amphipathic amine which readily crosses the cellular membrane via passive diffusion allowing it to directly access the intra-cellular and -organellar space. Within the cell, ambroxol acts as a weak base that becomes protonated and trapped in acidic subcellular compartments including lysosomes and other structures of the late endolysosomal pathway (Magalhaes J et al., 2018 supra; and Fois G et al., 2015 supra). These properties explain ambroxol's high bioavailability and tendency to accumulate in lipid-rich organ systems such as the brain, lungs, and skin (Mullin S et al., 2020 supra). Within the cytoplasm, ambroxol also acts as a potent scavenger and stabilizer of free radicals produced in the course of cellular metabolism and as part of the innate immune response (Stetinová, Herout and Kvetina, 2004 Clinical and Experimental Medicine, 4 (3), pp. 152-158). Finally, ambroxol has been shown to impart analgesia by directly blocking Na⁺ᵥ1.7/8 channels associated with neuropathic pain (Kern and Schwickert, 2017 supra).

Amphipathic trapping of ambroxol in acidic compartments exerts influence over the AENL system in several ways. One important consequence of the protonation of ambroxol in lysosomes is the resultant deacidification of the lysosomal lumen (Mullin S et al., 2020 supra, and Lu S et al., PloS One 12:20173771, 2017). This process mobilizes intraorganellar calcium stores which result in the activation of lysosome-associated transcription factor EB (TFEB). Activated TFEB relocalizes to the nucleus where it initializes transcription of a gene network with master regulatory control over lysosomal biogenesis (Medina D L et al., Nat Cell Biol 17:288-299, 2015). Upregulation of this gene network results in increased cellular capacity to degrade long-lived proteins (Sardiello et al., 2009 Science, 325 (5939), pp. 473-477), particularly proteins associated with neurodegenerative disorders such as hyperphosphorylated tau (Martini-Stoica et al., 2018 The Journal of Experimental Medicine, 215 (9), pp. 2355-2377). Ambroxol effectively activates TFEB both in cell culture as well as in vivo. In cultures derived from brain (Magalhaes et al., 2018 supra), skin (McNeill et al., 2014 supra), or immune-related tissues (Choi et al., 2018 Antimicrobial Agents and Chemotherapy, 62 (9)) application of ambroxol results in upregulation of TFEB at the transcription, protein, and/or activation levels.

Relatedly, ambroxol in a high dose regime can drive cellular autophagy into a secretory regime (see, for example, McNeill A et al., 2014 supra, and Silveira CRA et al., 2019 supra). Secretory autophagy comprises the mechanisms by which normally functioning and defective proteins are packaged into membrane-bound organelles termed autophagosomes and secreted extracellularly, and represents an alternative process to degradative autophagy in which the autophagosome fuses with a lysosome to drive breakdown and recycling of autophagosomal contents (Rabouille C et al., J Cell Sci 125:5251-5255, 2012). Upregulation of autophagy-dependent secretion is recognized as a potential route for disease modification in neurodegenerative disorders (Ponpuak M et al., Curr Opin Cell Biol 35:106-116, 2015). In the case of PD, as well as AD, aggregation-prone proteins aSyn (in PD; see, for example, Dehay B et al., J Neurosci Off J Soc Neurosci 30:12535-12544, 2010) and beta amyloid (in AD; see, for example, Vickers J C et al., Exp Neurol 141:1-11, 1996) form degradation-resistant inclusions that accumulate in autophagic vacuoles. In both cases, protein aggregates drive dysregulation at multiple points along the cellular autophagic processing pathways which may potentially be relieved by secretory unloading.

Ambroxol is capable of directly engaging the secretory autophagic system. In primary neuronal cultures from cells engineered to express PD pathology and unmodified counterparts, ambroxol enhances secretion of aSyn while clearing intracellular stores of aggregation-prone versions of this protein (Magalhaes J et al., 2018 supra). These results extend further to AD pathology, where clearance of intracellular aggregation-prone tau is driven by the application of bromhexine to brain-derived cell culture (Chauhan S et al., Nat Commun 6:8620, 2015). Taken together, these data indicate that ambroxol exerts beneficial effects on the AELN by increasing innate cellular degradative capacity while unloading toxic protein aggregates from cells such as neurons that possess lower innate degradative capabilities.

Another important system for human health ambroxol influences is the immune response. Epidemiological and preclinical work has indicated that driving the immune response away from an innate inflammatory response into an anti-inflammatory/adaptive state may be beneficial for multiple chronic disease states. For example, in many neurodegenerative disorders, longitudinal sampling of patient sera and cerebrospinal fluid indicate a progressive increase of inflammatory response markers, such as cytokines including interleukin 1β, 6, 8, and tissue necrosis factor alpha, and activation of the NLRP3-associated inflammasome that mirrors loss of cognitive function (Heneka et al., 2015 The Lancet. Neurology, 14 (4), pp. 388-405; Wang, Liu and Zhou, 2015 Translational Neurodegeneration, 4, p. 19). Concordantly, variants of many genes with innate immune regulatory function are associated with increased risk of AD and PD (Hollingworth et al., 2011 Nature Genetics, 43 (5), pp. 429-435; Griciuc and Tanzi, 2021 Current Opinion in Neurology, 34 (2), pp. 228-236). In contrast, upregulation of markers of anti-inflammation and adaptive immunity such as interferon gamma and interleukin 12 correlates with decreased incidence of AD in elderly adults (Yang et al., 2022 Alzheimer's & Dementia, 18 (4), pp. 645-653).

Neurodegenerative disorders are emblematic of chronic inflammatory states that arise when the immune system is unable to alleviate the source of immune challenge over a chronic timeframe. In the case of neurodegenerative disorders, these immune insults may in part be provided by toxic aggregated proteins produced largely by cells within the brain. Chronic inflammation can also occur in the context of infection that persists in the body such as mycoplasma pneumonia and inappropriate immune activation by self-derived antigens.

With respect to inflammation, ambroxol has also been shown to profoundly reshape the immune response to both pathogenic and self-derived threats. Studies across multiple organ systems, particularly the brain (Jiang et al., 2020 BioMed Research International, 2020, p. e8131286), lungs (Takeda et al., 2016 Immune Network, 16 (3), pp. 165-175; Zhang et al., 2016; Kókai et al., 2021 Microorganisms, 9 (4), p. 880), and gut (Schneider et al., 2021 EMBO Molecular Medicine, 13 (1), p. e12724; Cavalu et al., 2022 The FASEB Journal, 36 (9), p. e22496) have confirmed that ambroxol acts to reduce pro-inflammatory response to such threats while preserving the adaptive aspects of immune response. In particular, ambroxol has been shown to decrease expression of pro-inflammatory cytokines such as interleukins 1β, 6, 8, 10, and tissue necrosis factor-α (Bianchi et al., 1990 Agents and Actions, 31 (3-4), pp. 275-279; Jang et al., 2003 Pharmacology & Toxicology, 92 (4), pp. 173-179; Wang et al., 2011 Zhongguo Ying Yong Sheng Li Xue Za Zhi=Zhongguo Yingyong Shenglixue Zazhi=Chinese Journal of Applied Physiology, 27 (2), pp. 231-235) as well as reductions in pathways upstream of inflammasome activation such as NF-κB (Cavalu et al., 2022 supra). This suppression is due at least in part to ambroxol's ability to scavenge free radicals (Peroni et al., 2013 International Journal of Immunopathology and Pharmacology, 26 (4), pp. 883-887) and may also involve direct block of specific inflammation-associated channels (Schneider et al., 2021 supra).

Unlike non-specific immune suppressing agents such as non-steroidal anti-inflammatory drugs, ambroxol preserves and in some cases enhances aspects of the anti-inflammatory and adaptive immune system. This includes the upregulation of anti-inflammatory cytokines interleukins 10 and 12 as well as adaptive immune-associated interferon-γ in lung tissue in response to pathogen and ovalbumin challenge (Takeda et al., 2016 supra; Kókai et al., 2021 supra). These immune effectors may explain ambroxol's long-known clinical benefit in chronic respiratory diseases such as chronic obstructive pulmonary disorder (Plomer and de Zeeuw, 2017 MMW Fortschritte der Medizin, 159 (Suppl 5), pp. 22-33). Additionally, they may account for preclinical observations of reduced inflammation in models of ulcerative colitis (Schneider et al., 2021 supra) and reduced microglial activation in a model of intracerebral hemorrhage (Jiang et al., 2020 supra).

It is likely that the anti-inflammatory effects described above are related to ambroxol's impacts on autophagy. Autophagy has been noted to have a key role in regulating immune response. Particularly, activation of TFEB function drives degradation of key mediators of inflammation including inflammasome components such as NLRP3 and ASC (Shi et al., Nature Immunology, 13 (3), pp. 255-263 (2012); Deretic, Immunity, 54 (3), pp. 437-453 (2021). Inflammasome inactivation through degradation results in dampened inflammatory response including reduction in production and release of pro-inflammatory cytokines such as IL-1B. It is thus probable that ambroxol's immune modulatory function lies downstream of its effect on autophagy.

Finally, ambroxol possesses potent analgesic properties. Mechanistically, this effect is attributed to ambroxol's ability to block voltage-dependent sodium channels including Naᵥ1.7 (Leffler, Reckzeh and Nau, 2010 European Journal of Pharmacology, 630 (1-3), pp. 19-28) and 1.8 (Weiser and Wilson, 2002 Molecular Pharmacology, 62 (3), pp. 433-438) which are preferentially expressed by neurons involved in nociception (Bennett et al., 2019 Physiological Reviews, 99 (2), pp. 1079-1151). This mechanism at least partially explains ambroxol's classic use as a treatment for moderate to severe pain associated with acute sore throat (Fischer et al., 2002 Arzneimittel-Forschung, 52 (4), pp. 256-263). It additionally has increased interest in the use of ambroxol for the treatment of neuropathic pain (Russo et al., 2022 supra).

Thus, in conclusion, and while not wishing to be bound by theory, it is considered that the abovementioned observations indicate that ambroxol may increase degradation and/or secretion of toxic intracellular proteins, protein fragments, misfolded proteins, protein aggregates or debris associated with disorders of the AELN. Ambroxol further modulates immune response to a less inflammatory state through likely through scavenging of free radicals and interactions with inflammation-associated channels. Finally, ambroxol directly blocks channels upregulated in disease states characterized by chronic pain.

Implications—Ambroxol and related compounds (including bromhexine and pharmaceutically-acceptable salts of ambroxol such as ambroxol hydrochloride), as well as ambroxol analogs such as the polydeuterated analogs of the invention, offer considerable potential as an effective treatment of neurological diseases and conditions such as PD, AD and other aging-associated diseases involving dysfunction of autophagy), by enabling the secretion of toxic aggregated proteins (e.g., aggregated aSyn, beta amyloid and tau protein) from affected cells despite autophagy dysfunction, and by maintaining organelle health.

In some cases, it may be advantageous to administer ambroxol (or a related compound or polydeuterated analog of the invention) in a combination therapy with an agent intended for, for example, the prevention and/or removal of toxic aggregated proteins.

Thus, in another aspect of the invention, a method is provided for preventing, reducing the symptoms of and/or stabilizing the progression of Alzheimer's disease (AD) or other diseases associated with pathological protein misfolding, aggregation and deposition (including Parkinson's disease (PD), Huntingdon's disease (HD) and Frontotemporal degeneration (FTD)) in a subject, said method comprising administering to the subject an effective amount of ambroxol (or a related compound such as ambroxol hydrochloride and bromhexine) or a polydeuterated analog of the invention in combination with one or more suitable anti-beta amyloid antibody or fragment thereof.

Such a combination therapy may produce, for example, a synergistic effect in removing aggregated beta amyloid associated with, for example, AD.

The ambroxol (or related compound) or polydeuterated analog of the invention and the anti-beta amyloid antibody or fragment thereof may be administered, for example, in the same pharmaceutical composition or in separate pharmaceutical compositions. If administered in separate pharmaceutical compositions, the ambroxol (or related compound) or polydeuterated analog of the invention and the anti-beta amyloid antibody or fragment thereof may be administered simultaneously or sequentially in any order (e.g., within seconds or minutes or even hours (e.g., 2 to 48 hours)).

The anti-beta amyloid antibody or fragment thereof may be selected from those known to those skilled in the art. Suitable antibodies may include human or humanized anti-Aβ monoclonal antibodies of the group consisting of Bapineuzumab (Pfizer Inc./Janssen Pharmaceuticals, Inc.), Solanezumab (Eli Lilly and Company), Gantenerumab (Hoffman-La Roche), Crenezumab (Genentech, Inc.), Ponezumab (Pfizer Inc.), BAN2401/Lecanamab (BioArctic Neuroscience, AB/Eisai Co., Ltd/Biogen, Inc.) and Aducanumab (Aduhelm™ Biogen, Inc.) (see also, van Dyck C H., Biol Psychiatry 83 (4): 311-319, 2018). Suitable antibody fragments may include fragments such as Fab fragments and scFv antibodies targeted to Aβ including scFv molecules described by Sebollela A., J Neurochem 142 (6): 934-937, 2017, and Zha J et al., Scientific Reports 6:36631, 2016.

In some embodiments, the method is to be operated for the prevention of, reduction of symptoms of, and/or for slowing the progression of, AD or other diseases associated with pathological protein misfolding, aggregation and deposition (including Parkinson's disease (PD), Huntingdon's disease (HD) and Frontotemporal degeneration (FTD)). Thus, the method may, for example, prevent the occurrence, development or progression of the disease or condition, or the occurrence, development or progression of one or more symptom or deleterious characteristic of the disease or condition (e.g., the toxic aggregation of Aβ proteins). Aggregation of beta amyloid and tau are known to appear early in the development of Alzheimer's disease, so a therapeutic method intended to prevent and/or remove toxic aggregates of Aβ or tau, as may be achieved by administering ambroxol (or related compound) or polydeuterated analog of the invention, offers significant potential.

In a variation, the invention may provide a method for the prevention of, reduction of symptoms of, and/or for slowing the progression of, AD or other diseases associated with pathological protein misfolding, aggregation and deposition (including Parkinson's disease (PD), Huntingdon's disease (HD) and Frontotemporal degeneration (FTD)), comprising administering to the subject an effective amount of ambroxol (or a related compound such as ambroxol hydrochloride and bromhexine) or a polydeuterated analog of the invention. That is, the ambroxol or related compound or polydeuterated analog of the invention may be used as the sole active agent.

In some embodiments of the methods for the prevention of, reduction of symptoms of, and/or the slowing of the progression of, AD or other diseases associated with pathological protein misfolding, aggregation and deposition (including Parkinson's disease (PD), Huntingdon's disease (HD) and Frontotemporal degeneration (FTD)), the subject may be selected on the basis of a suitable biomarker indicative of an at-risk patient or patient in an early stage of development of AD or other diseases associated with pathological protein misfolding, aggregation and deposition (including Parkinson's disease (PD), Huntingdon's disease (HD) and Frontotemporal degeneration (FTD)). For AD, the biomarker may be certain phosphorylated tau proteins (p-tau) that have been found to be present in the CSF and/or blood at increased level(s) in early or preclinical stage(s) of AD (Suárez-Calvet M et al., EMBO Mol Med 12: e12921, 2020). Thus, in one example, the methods may further comprise selecting the subject by assaying for an increased level of p-tau217 (ie tau phosphorylated at the Thr-217 residue) or, more preferably, p-tau181 (ie tau phosphorylated at the Thr-181 residue), in a suitable sample (eg a sample of CSF, whole blood or plasma). These biomarkers can accurately differentiate Aβ-positive from Aβ-negative cognitively unimpaired subjects, and moreover, it has been shown that the p-tau181 biomarker is mildly but significantly increased in the preclinical stage of AD (Suárez-Calvet M et al., 2020 supra). Other p-tau tau isoforms may be assayed as well, such as p-tau231 (Ashton, N. J. et al, Acta Neuropathol 1-16 (2021) or p-tau235 (Lantero-Rodriguez, J. et al. Embo Mol Med 13, e15098 (2021)). Other potential biomarkers include glucose metabolism, or aggregated proteins such as beta amyloid or Tau measured by Positron Emission Tomography (PET) (Therriault, J. et al. Nat Aging 1-10 (2022) doi: 10.1038/s43587-022-00204-0. Iaccarino, L., et al. “Journal of Alzheimer's Disease, 59, pp 603-614 (2017).) Retinal-imaging based biomarkers including amyloid deposition and thinning of the nerve fiber layer and could also be used for the early diagnosis of AD (Snyder, P. J. et al. Alzheimers Dement. Diagn. Assess. Dis. Monit. 4, 169-178 (2016); Koronyo, Y. et al. JCI Insight 2, (2017)).

Other suitable biomarkers indicative of an at-risk patient or patient in an early stage of development of AD or other amyloidoses include, for example, 24 allele of the ApoE gene and/or the pyroglutamate-modified amyloid β protein, N3pG. Plasma NFL (Neurofilament light chain) is elevated in many neurodegenerative diseases. It is not specific to any specific neurodegenerative diseases, and instead reflects disease activity and severity. For example it is elevated in Multiple Sclerosis patients who have active ongoing demyelination, but drops when the patients are treated with immunosuppressants (Barro, C, & Zetterberg, H. Acta Neurol Scand (2021) doi: 10.1111/ane.13415). Plasma exosomes bearing neuronal proteins also appear promising biomarkers to diagnose a number of neurodegenerative diseases, including Parksinon's disease, Alzheimer's disease, and head injury (Rastogi, S. et al. Int J Mol Sci 22, 440 (2021)).

In some embodiments of the methods for the prevention of, reduction of symptoms of, and/or the slowing of the progression of, AD or other diseases associated with pathological protein misfolding, aggregation and deposition (including Parkinson's disease (PD), Huntingdon's disease (HD) and Frontotemporal degeneration (FTD)), the subject may be selected on the basis of genotyping of at least one gene or locus indicative of a patient at-risk of AD or other diseases associated with pathological protein misfolding, aggregation and deposition (including Parkinson's disease (PD), Huntingdon's disease (HD) and Frontotemporal degeneration (FTD)). For AD, the genotyping may be of the gene encoding apolipoprotein E (ApoE); subjects carrying the 24 allele of ApoE are at increased risk of AD compared with those carrying the more common 83 allele, whereas the 22 allele decreases risk (Liu C-C et al., Nat Rev Neurol. 9 (2): 106-118, 2013). Suitable methodologies for conducting ApoE genotyping are known to those skilled in the art and include, for example, suitable PCR protocols (see, for example, Zhong L et al., Mol Neurodegener. 11:2, 2016). However, in other embodiments, the subject may be selected on the basis of genotyping of p-tau217, p-tau181 and/or N3pG.

In some embodiments of the methods for preventing and/or treating Alzheimer's disease (AD) or other diseases associated with pathological protein misfolding, aggregation and deposition (including Parkinson's disease (PD), Huntingdon's disease (HD) and Frontotemporal degeneration (FTD)), it may be preferred that the ambroxol (or related compound) or polydeuterated analog of the invention is administered to the subject in a relatively high daily dosage selected from a dosage that:
(i) provides a peak concentration in serum of the subject that is greater than 1 μM such as, for example, 2-50 μM, 2-25 μM or 10-20 μM;
(ii) provides a peak concentration in brain tissue of the subject that is greater than 3 M such as, for example, 5-50 μM, 5-25 μM or 10-20 μM; or
(iii) is in the range of about 250 mg-1000 mg/day or 750-1000 mg/day; since such a daily dose may be necessary to enable the ambroxol (or related compound) or polydeuterated analog of the invention to upregulate the secretion of toxic aggregated proteins (e.g., aggregated Aβ) from affected cells.

### Pharmaceutical Compositions

In an aspect, the invention includes a composition comprising a therapeutically effective amount of a compound of formula I (for example, a compound as shown in FIG. 1) or a pharmaceutically acceptable salt, solvate or prodrug thereof, in conjunction with a pharmaceutically acceptable carrier for preventing and/or treating various diseases and medical conditions in a subject including respiratory diseases and conditions, lysosomal storage disorders (LSDs), and neurological diseases and conditions, or for extending life expectancy of a subject. For example, a composition comprising a compound of formula I (or a pharmaceutically acceptable salt, solvate or prodrug thereof) may be used for (a) treating, inhibiting, or reducing aging of a subject, (b) treating, inhibiting, or reducing an age-related symptom or an age-related disease in a subject, and/or (c) increasing the healthspan, lifespan, and/or mental acuity of a subject.

The compound of formula I (or a pharmaceutically acceptable salt, solvate or prodrug thereof) may be administered in the form of a pharmaceutical composition, in combination with a pharmaceutically acceptable carrier. In such compositions, the compound of formula I may comprise from 0.1 to 99.99 weight percent.

The compound of formula I is preferably administered with a pharmaceutically acceptable carrier selected on the basis of the selected route of administration and standard pharmaceutical practice. The compound of formula I may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa. Suitable dosage forms may comprise, for example, tablets, capsules, solutions, parenteral solutions, troches, suppositories, or suspensions. Suitable examples of the preparation of oral, topical, suppository and parenteral formulations of ambroxol, bromhexine, or other ambroxol derivatives, which may be readily adapted for compounds of the present invention, are disclosed in, for example, Examples 1-8 of WO 2005/007146, or its equivalent US 2005/00148747, incorporated herein by reference.

In another aspect, the invention provides the use of a compound of formula I in the preparation of a medicament for preventing and/or treating various diseases and medical conditions in a subject including respiratory diseases and conditions, lysosomal storage disorders (LSDs), and neurological diseases and conditions, or for extending life expectancy of a subject.

For parenteral administration, the compound of formula I may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol, or with a plant extract as a supplement. Solutions for parenteral administration preferably contain a water-soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or non-aqueous solution, dispersion, suspension or emulsion.

For oral administration, the compound of formula I (or a pharmaceutically acceptable salt, solvate or prodrug thereof) may be combined with one or more solid inactive ingredients for the preparation of tablets, capsules, pills, powders, granules or other suitable oral dosage forms. For example, the compound of formula I may be combined with at least one excipient such as fillers, binders, humectants, disintegrating agents, solution retarders, absorption accelerators, wetting agents, absorbents or lubricating agents. According to one tablet embodiment, the compound of formula I may be combined with carboxymethylcellulose calcium, magnesium stearate, mannitol and starch, and then formed into tablets by conventional tableting methods.

For oral administration, the compound of formula I (or a pharmaceutically acceptable salt, solvate or prodrug thereof) may be provided in a liquid oral pharmaceutical composition. Liquid oral dosage forms can offer unique advantages over solid dosage forms like tablets and capsules. For example, in many instances, the amount of active ingredient necessary to treat a specific disease or condition requires multiple tablets or capsules one or more times a day. The ingestion of multiple tablets or capsules includes not only the active pharmaceutical ingredient, but multiple doses of excipients which are used to formulate the tablets and capsules. Such excipients, such as oils and alcohols, are typically not well tolerated by many patients and commonly lead to gastric distress. Additionally, liquid oral dosage forms are more patient compliant than solid dosage forms since delivery of the active pharmaceutical ingredient is achieved in only one or two doses per day. Further, liquid oral dosage forms provide rapid absorption of an active pharmaceutical ingredient from the gastro-intestinal tract. Moreover, liquid oral dosage forms allow the use of flavoring and/or palatability agents, which further promotes patient acceptance and compliance.

Accordingly, in some preferred embodiments, the composition of the invention will be a liquid oral pharmaceutical composition. Such a composition may be particularly suited for preventing and/or treating a lysosomal storage disorder (LSD) such as Gaucher's disease, or a neurological disease or condition such as PD. In some embodiments, the composition will comprise a “high loading” of the compound of formula I (or a pharmaceutically acceptable salt, solvate or prodrug thereof) in order to deliver an effective amount in a small volume (e.g., 15 mL dose, two or more times a day). A high drug loading liquid oral pharmaceutical composition according to the invention may provide at least one of the following advantages: (1) improved adsorption in the gastrointestinal tract; (2) maintain effective blood concentration over a 24 hour period; (3) reduced undesirable side effects of excipients as compared to solid dosage forms; (4) a reduction in the number of doses required; and (5) an improved taste and mouthfeel qualities.

In one specific embodiment of a high drug loading liquid oral pharmaceutical composition of the invention, the composition comprises (i) a compound of formula I (or a pharmaceutically acceptable salt, solvate or prodrug thereof); and (ii) at least one pharmaceutically acceptable excipient, wherein the compound of formula I (or a pharmaceutically acceptable salt, solvate or prodrug thereof) is in the form of granules having a granular core comprising from about 60 to about 97 weight percent of an active pharmaceutical ingredient and from about 3 to about 40 weight percent of the excipient, wherein the weight percent is based on the total weight of the granular core.

In another specific embodiment of a high drug loading liquid oral pharmaceutical composition of the invention, the composition comprises (i) a compound of formula I (or a pharmaceutically acceptable salt, solvate or prodrug thereof); (ii) at least one pharmaceutically acceptable excipient; and (iii) a diluent, wherein the compound of formula I (or a pharmaceutically acceptable salt, solvate or prodrug thereof) is in the form of granules having a granular core comprising from about 60 to about 97 weight percent of an active pharmaceutical ingredient and from about 3 to about 40 weight percent of the excipient, wherein the weight percent is based on the total weight of the granular core; wherein the granule core is coated with (iv) a water-soluble seal coating in an amount to provide from about 0.5 to about 5 percent weight gain, and (v) an enteric coating in an amount to provide from about 0.5 to about 50 percent weight gain.

Such high drug loading liquid oral pharmaceutical compositions may be prepared by a method comprising, for example: (a) preparing granules having a granular core comprising from about 60 to about 97 weight percent of a compound of formula I (or a pharmaceutically acceptable salt, solvate or prodrug thereof) and from about 3 to about 40 weight percent of at least one pharmaceutically acceptable excipient, wherein the weight percent is based on the total weight of the granular core; (b) coating the granules with a water-soluble seal coating in an amount to provide from about 0.5 to about 5 percent weight gain; (c) coating the granules prepared in step (b) with an enteric coating in an amount to provide from about 0.5 to about 50 percent weight gain; and (d) preparing a liquid suspension comprising the enteric coated granules prepared in step (c) and a liquid suspension formulation, wherein the liquid suspension formulation comprises a suspending agent, a vehicle to enhance the stability of the high drug loading liquid oral pharmaceutical composition, and a diluent.

The compound of formula I (or a pharmaceutically acceptable salt, solvate or prodrug thereof) is preferably in the form of granules which preferably, after micronizing, have a particle size from about 100 microns to about 500 microns, more preferably, from about 100 to about 300 microns, from about 150 to about 350 microns, from about 200 to about 350 microns, and even more preferably, the particle size is from about 100 to about 200 microns, from about 150 to about 250 microns, from about 200 to about 300 microns, from about 200 to about 400 microns, from about 250 to about 350 microns, from about 250 to about 450 microns, from about 300 to about 400 microns, from about 300 to about 500 microns, from about 350 to about 450 microns, and/or from about 400 to about 500 microns in size. It has been found that particle sizes within these ranges are coatable and provide ease of swallowing without urging the user to bite down. For example, it has been found that in preferred embodiments, a particle size of from about 250 to about 350 microns balances the ability to coat with the ability to avoid the gritty “mouth feel”. The particle size can be determined by laser light scattering for instance using a Malvern Mastersizer Apparatus MS 2000 equipped with a Hydro S dispersion unit. Micronization of compounds of formula I, for example, may be performed in the dry state using dry mills such as cutting mills, pin/cage mills, hammer mills, jet mills, fluidized bed jet mills and ball mills.

A preferred excipient that may be used to prepare the granular core is a binder. The binder may be any water soluble pharmaceutically acceptable polymer. In one embodiment, the compound of formula I (or a pharmaceutically acceptable salt, solvate or prodrug thereof) is in the form of a powder which the binder necessarily bonds together due to the poor cohesive properties of most powders. Preferably, the binder is selected from povidone (polyvinylpyrrolidone), copovidone (vinylpyrrolidone-vinylacetate copolymer), microcrystalline cellulose, powdered cellulose, crystalline cellulose, siliconized microcrystalline cellulose, cellulose derivatives such as hydroxymethylcellulose, hydroxyethylcellulose, hydroxypropylcellulose and hydroxypropylmethylcellulose, starch, pregelatinized starch, polymethacrylates, compressible sugars, sucrose and sugar alcohols such as mannitol, sorbitol, maltitol and xylitol and mixtures thereof. More preferably, the binder is hydroxypropyl cellulose (Klucel LF).

The granules used in the high loading liquid oral pharmaceutical composition are preferably prepared by direct spheronization involving preparing a granular core comprising from about 60 to about 97 weight percent of a compound of formula I (or a pharmaceutically acceptable salt, solvate or prodrug thereof), preferably from about 75 to about 97 weight percent, and more preferably from about greater than 90 weight percent to 97 weight percent, and from about 3 to about 40 weight percent of a binder, preferably from about 3 to about 25 weight percent, and more preferably from about 3 to about 10 weight percent, wherein the weight percent is based on the total weight of the granular core. The granulation can be carried out under high shear (mixer granulation) or in a fluidized bed (fluidized bed granulation).

The granular cores are optionally seal coated with a water-soluble seal coating in an amount to provide from about 0.5 to about 5 percent weight gain, and more preferably from about 0.5 to about 3 percent weight gain, and even more preferably from about 0.5 to about 2 percent weight gain, and even more preferably from about 0.5 to about 1 percent weight gain, and even more preferably from about 1 to about 2 percent weight gain, and even more preferably from about 1 to about 3 percent weight gain, and still even more preferably from about 1 to about 4 percent weight gain, and more preferably from about 2 to about 3 percent weight gain, and even more preferably from about 2 to about 4 percent weight gain, and more preferably about 1 percent weight gain, and more preferably about 2 percent weight gain, and more preferably about 3 percent weight gain, and/or more preferably about 4 percent weight gain. It has been found that applying a water-soluble seal coating generates a smooth and uniform granule which is more receptive to receiving an enteric coating. Preferred water-soluble polymers include hydroxypropylmethylcellulose (HPMC), carboxymethylcellulose (sodium and calcium salts), ethylcellulose, methylcellulose, hydroxyethylcellulose, ethyl hydroxyethylcellulose, hydroxypropylcellulose (HPC), L-HPC (low-substituted HPC), polyvinylpyrrolidone, polyvinyl alcohol, polymers of acrylic acid and its salts, vinylpyrrolidone-vinyl acetate copolymers (for example Kollidon® VA64, BASF), gelatine, guar gum, partially hydrolysed starch, alginates and xanthan. Most preferably, the water-soluble polymer is hydroxypropylmethyl cellulose. The seal coating is preferably applied by means of a bottom spray fluidized bed coater equipped with a Wurster column.

In addition to the seal coating or in the absence of a seal coating, the granular cores may optionally be coated with an enteric coating directly on the granular core or on a seal coating previously applied to the granular cores. Preferably, the enteric coating is applied in an amount to provide from about 0.5 to about 50 percent weight gain, from about 1 to about 40 percent weight gain, from about 2 to about 30 percent weight gain, from about 3 to about 20 percent weight gain, or from about 4 to about 10 percent weight gain, based on the total weight of the granular core. More preferably, the enteric coating is applied in an amount to provide from about 0.5 to about 5 percent weight gain, from about 1 to about 4 percent weight gain, or from about 2 to about 3 percent weight gain, based on the total weight of the granular core. The enteric coating is preferably applied by means of a bottom spray fluidized bed coater equipped with a Wurster column.

The enteric coating comprises a polymer selected from an acrylate polymer or an aqueous cellulose dispersion. Combinations of acrylate polymers and/or aqueous cellulose dispersions may also be used. Preferably, the acrylate polymer is selected from polymethacrylate methylmethacrylate copolymer (Eudragit® L-100), polyethylacrylate methylmethacrylate trimethylammonioethyl methacrylate chloride copolymer (Eudragit RL-100, RS-100), polymethacrylate ethylacrylate copolymer (Eudragit® L30D-55), ethylacrylate methylmethacrylate trimethylammonioethyl methacrylate chloride copolymer (Eudragit® RL30D), ethylacrylate methylmethacrylate trimethylammonioethyl methacrylate chloride copolymer (Eudragit® RS30D), and polyethylacrylate methylmethacrylate copolymer (Eudragit® NE30D). More preferably, the acrylate polymer is polymethacrylate ethylacrylate copolymer (Eudragit® L30D-55). The enteric coating may be preferably applied by means of a bottom spray fluidized bed coater equipped with a Wurster column. The enteric coating may increase delivery of the active pharmaceutical ingredient to a region of the gastrointestinal tract of a subject in which the pH is between about 4.5 and about 6.5. The enteric coating also increases delivery of the compound of formula I (or a pharmaceutically acceptable salt, solvate or prodrug thereof) to the proximal or mid-small intestine or both. In addition, the enteric coating may increase delivery of the compound of formula I (or a pharmaceutically acceptable salt, solvate or prodrug thereof) to one or more of the duodenum, jejunum, or mid-ileum. Preferably, the enteric coating begins to dissolve in an aqueous solution at pH between about 4.5 to about 5.5.

The vehicle to enhance the stability of the high drug loading liquid oral pharmaceutical composition may preferably be a protective colloid.

The high loading liquid oral pharmaceutical composition of the invention may additionally contain a plasticizer. Preferred plasticizers are diethyl phthalate, dibutyl phthalate, triethyl citrate, and glycerol. A combination of plasticizers may also be used. More preferably, the plasticizer is triethyl citrate.

In one embodiment, the high loading liquid oral pharmaceutical composition is in the form of a solution. In another embodiment, the high loading liquid oral pharmaceutical composition is in the form of a suspension. To form a suspension, the enteric coated granules may be combined with a liquid suspension formulation. The liquid suspension formulation may comprise a suspending agent, a vehicle to enhance the stability of the high drug loading liquid oral pharmaceutical composition, and a diluent. The suspending agent and the vehicle to enhance the stability of the high drug loading liquid oral pharmaceutical composition may be the same or different since many suspending agents also serve as stability enhancers.

Examples of suspending agents include, but are not limited to, methylcellulose, sodium carboxymethylcellulose, hydroxyethylcellulose, hydroxypropylmethylcellulose, gum tragacanth, and glycerol monostearate. More preferably, the vehicle to enhance the stability of the liquid oral pharmaceutical composition is a protective colloid. Examples of protective colloids include, but are not limited to, hydroxymethylcellulose, carboxymethylcellulose sodium, polyvinylalcohol, gelatin, and polyvinyl pyrrolidone. A combination of protective colloids may also be used. A preferred protective colloid is a mixture of cellulose gum, xanthan gum, and carrageenan. Examples of diluents include, but are not limited to, water, alcohols (such as for example methyl alcohol, ethyl alcohol, propyl alcohol, i-propyl alcohol etc.), acetone, glycerin, oils (such as for example castor oil), any other pharmaceutically acceptable diluent or mixtures thereof. Most preferably, water is used as the suspension or solution diluent. In addition, a pH modifier and/or an antioxidant may also be used.

In some specific embodiments of a liquid suspension formulation of the invention, the formulation includes: microcrystalline cellulose and carboxymethylcellulose sodium (Avicel RC-591), gum (CP Kelco), and water; or microcrystalline cellulose and carboxymethylcellulose sodium (Avicel RC-591), cellulose gum, xanthan gum, and carrageenan (Ticaloid Ultrasmooth), and water.

In accordance with a preferred embodiment, the liquid suspension formulation includes from about 0.5% to about 3% of at least one suspending agent; from about 0.5% to about 1% of at least one protective colloid; and about 98% of a diluent, wherein the weight percent values are based on the weight of the liquid suspension formulation. More preferably, the protective colloid will be used in an amount of about 0.1 to 0.5 weight percent. In some specific embodiments, the liquid suspension formulation includes about 1.5% of at least one suspending agent; from about 0.2% of at least one protective colloid; and about 98% of a diluent.

Preferably, the high drug loading liquid oral pharmaceutical composition in suspension form has a viscosity less than about 5 Pa s. More preferably, high drug loading liquid oral pharmaceutical composition in suspension form has a viscosity less than about 3 Pas, and most preferably, less than about 1 Pa s.

In some embodiments, the high drug loading liquid oral pharmaceutical composition is used to prevent and/or treat a lysosomal storage disease selected from: Gaucher's disease (including Type 1, Type 2 and Type 3 Gaucher's disease), Pompe disease (including infantile and late-onset forms) and Fabry disease, or to prevent and/or treat (e.g., alleviate the symptoms) of PD. Other lysosomal storage diseases that may be prevented and/or treated with the high drug loading liquid oral pharmaceutical composition include: GMI-gangliosidosis, Tay-Sachs disease, Sandhoff disease, Niemann-Pick disease, Krabbe disease, Farber disease, Metachromatic leukodystrophy, Hurler-Scheie disease, Hunter disease, Sanfilippo disease A, Sanfilippo disease B, Sanfilippo disease C, Sanfilippo disease D, Morquio disease A, Morquio disease B, Maroteaux-Lamy disease, Sly disease alpha-Mannosidosis beta-Mannosidosis, Fucosidosis, Sialidosis and Schindler-Kanzaki disease.

In some more specific embodiments, the high drug loading liquid oral pharmaceutical composition is used to prevent and/or treat a lysosomal disease selected from Type 1, Type 2 and Type 3 Gaucher's disease. In some further specific embodiments, the high drug loading liquid oral pharmaceutical composition is used to prevent and/or treat a subject with a mutation in a glucocerebrosidase (GCase) (e.g., a mutation in a beta glucocerebrosidase, wherein the subject may also have either Gaucher's and/or Parkinson's Disease). The mutation in beta glucocerebrosidase may be selected from:
a. A point mutation at any one of the following positions: D140H, V15L, G46E, K79N, R119Q, P122S, R131L, K157Q, N188S, Y212H, F213I, F216V, F216Y, H225Q, F251L, R257E, P289L, A309V, H311R, W312C, Y323I, G325R, E326K, C₃₄₂G, R353G, R359X (termination), S364T, N370S, L371V, G377S, V394L, V398F, P401L, D409H, D409V, P415R, L444P, A456P, V460V, R463C, G478S, or R496H and/or any combination thereof;
b. A point mutation at L444P;
c. A point mutation at N370S;
d. A point mutation at E326K;
e. Point mutations at L444P, A456P, and V460V;
f. Point mutations at D140H and E326K;
g. Point mutations at H255Q and D409H;
h. A guanine insertion at 84GG;
i. A splice site mutation in intron 2 (IVS2DS+1G-A), resulting in the skipping of exon 2;
j. A 1-bp deletion (1023delC in the genomic sequence) in the GCase gene,
k. A55-bp deletion (nucleotides 5879-5933 in genomic DNA) in the GCase gene;
l. A homozygous 259C-T transition (1763 in the genomic DNA);
m. A homozygous 1-bp deletion in the GCase gene, resulting in a frameshift and premature truncation of the protein in exon 6; and
n. A G-to-A substitution at the first position in the splice site of intron 10 of the GCase gene, resulting in the insertion of the first 11 base pairs of IVS10 and deletion of the first 11 base pairs of exon 11.

In some embodiments, the high drug loading oral liquid pharmaceutical composition may be given to a subject who is also receiving enzyme replacement therapy (e.g., combination therapy). Examples of such enzyme replacement therapy include, but are not limited to recombinant glucocerebrosidase, such as, for example, Imiglucerase, Velaglucerase, Taliglucerase alfa (ELELYSO®), and/or Eliglustat (CERDELGA®). The high drug loading oral liquid pharmaceutical composition may be administered simultaneously, sequentially or at different times with the enzyme replacement therapy.

In some embodiments, the high drug loading oral liquid pharmaceutical composition comprises a polydeuterated analog of ambroxol or a polydeuterated analog of bromhexine or a pharmaceutically acceptable salt thereof for treating a subject having a misfolded and/or erroneously transported glucocerebrosidase. In further embodiments, the high drug loading oral liquid pharmaceutical composition comprises a polydeuterated analog of ambroxol or a polydeuterated analog of bromhexine or a pharmaceutically acceptable salt thereof for treating or preventing a lysosomal storage disorder in a subject. In yet further embodiments, the high drug loading oral liquid pharmaceutical composition comprises a polydeuterated analog of ambroxol or a polydeuterated analog of bromhexine or a pharmaceutically acceptable salt thereof for treating a subject having a mutation in a glucocerebrosidase. In yet still further embodiments, the high drug loading oral liquid pharmaceutical composition comprises a polydeuterated analog of ambroxol or a polydeuterated analog of bromhexine or a pharmaceutically acceptable salt thereof for treating a subject having a mutation in a beta-glucocerebrosidase. Also, in some preferred embodiments, the mutation in beta-glucocerebrosidase is selected from N370S, L444P, and/or E326K. In a further embodiment, the high drug loading oral liquid pharmaceutical composition comprises a polydeuterated analog of ambroxol or a polydeuterated analog of bromhexine or a pharmaceutically acceptable salt thereof for treating a subject suffering from Gaucher's disease. In a further embodiment, the high drug loading oral liquid pharmaceutical composition comprises a polydeuterated analog of ambroxol or a polydeuterated analog of bromhexine or a pharmaceutically acceptable salt thereof for treating a subject suffering from PD.

The pharmaceutical composition of the invention may also be formulated in a unit dosage form, each dosage containing from about 50 to about 1000 mg, more typically, about 250 to about 500 mg of the compound of formula I per unit dosage. The term “unit dosage form” refers to physically discrete units suitable as a unitary dosage for human subjects and other mammals, each unit containing a predetermined quantity of the compound of formula I calculated to produce the desired therapeutic effect, in association with a suitable pharmaceutically acceptable carrier.

In further preferred embodiments, the compound of formula I (or a pharmaceutically acceptable salt, solvate or prodrug thereof) is administered as several doses over a given period of time, e.g., a daily dose for a week or more. For example, a daily dosage from about 20-500 mg/day, 50-150 mg/day, 50-200 mg/day, 50-250 mg/day, 250-500 mg/day, or 250 mg-1000 mg/day may be utilized. Higher or lower doses are also contemplated as it may be necessary to use dosages outside these ranges in some cases. The daily dosage may be divided, such as being divided equally into two to four times per day daily dosing.

The pharmaceutical composition of the invention may also be formulated so as to provide slow or controlled release of the active ingredient therein using, for example, hydroxypropylmethylcellulose in varying proportions to provide the desired release profile, other polymer matrices, gels, permeable membranes, osmotic systems, multilayer coatings, microparticles, liposomes and/or microspheres.

In general, a controlled-release preparation is a pharmaceutical composition capable of releasing the active ingredient at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations.

U.S. Pat. No. 5,674,533 discloses controlled-release pharmaceutical compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 describes a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 describes controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. Biodegradable microparticles are known for use in controlled-release formulations. U.S. Pat. No. 5,354,566 discloses a controlled-release powder that contains the active ingredient. U.S. Pat. No. 5,733,566, describes the use of polymeric microparticles that release antiparasitic compositions. Any or all of these techniques may be adapted for the controlled release of compounds of formula I (or a pharmaceutically acceptable salt, solvate or prodrug thereof).

The controlled release of the compound of formula I (or a pharmaceutically acceptable salt, solvate or prodrug thereof) may also be stimulated by various inducers, for example pH, temperature, enzymes, water, or other physiological conditions or compounds. Various mechanisms of drug release exist. For example, in one embodiment, the controlled-release component may swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the compound of formula I (or a pharmaceutically acceptable salt, solvate or prodrug thereof) in the pharmaceutical composition. In another embodiment, the controlled-release component is biodegradable, induced by exposure to the aqueous environment, pH, temperature, or enzymes in the body. In another embodiment, sol-gels may be used, wherein the active ingredient is incorporated into a sol-gel matrix that is a solid at room temperature. This matrix is implanted into a subject, preferably a human or other mammal, having a body temperature high enough to induce gel formation of the sol-gel matrix, thereby releasing the active ingredient into the subject.

Compositions comprising compounds of the invention that are suitable for administration intranasally or by inhalation are of particular interest. As such, the compound of formula I may be formulated for administration intranasally or by inhalation, typically in the form of a dry powder (either alone, as a mixture, for example, in a dry blend with lactose in anhydrous or monohydrate form, preferably monohydrate, mannitol, dextran, glucose, maltose, sorbitol, xylitol, fructose, sucrose or trehalose, or as a mixed component particle, for example, mixed with phospholipids) from a dry powder inhaler or as an aerosol spray from a pressurized container, pump, spray, atomizer (preferably an atomizer using electrohydrodynamics to produce a fine mist), or nebulae, with or without the use of a suitable propellant, such as dichlorofluoromethane.

The pressurized container, pump, spray, atomizer, or nebulae contains a solution or suspension of the compound of formula I (or a pharmaceutically acceptable salt, solvate or prodrug thereof) comprising, for example, ethanol (optionally, aqueous ethanol) or a suitable alternative agent for dispersing, solubilizing or extending release of the compound, the propellant(s) as solvent and an optional surfactant, such as sorbitan trioleate or an oligolactic acid.

Prior to use in a dry powder or suspension formulation, the compound of formula I (or a pharmaceutically acceptable salt, solvate or prodrug thereof) is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

A suitable solution formulation of the compound of formula I (or a pharmaceutically acceptable salt, solvate or prodrug thereof) for use in an atomizer using electrohydrodynamics to produce a fine mist, may contain from 1 μg to 20 mg of the compound per actuation and the actuation volume may vary from 1 μL to 100 μL. A typical formulation may comprise the compound of formula I, propylene glycol, sterile water, ethanol and sodium chloride. Alternative solvents which may be used instead of propylene glycol, include glycerol and polyethylene glycol. Capsules, blisters and cartridges (made, for example, from gelatin or HPMC) for use in an inhaler or insufflator may be formulated to contain a powder mix of the compound of formula I (or a pharmaceutically acceptable salt, solvate or prodrug thereof), a suitable powder base such as lactose or starch and a performance modifier such as L-leucine, mannitol, or magnesium stearate.

Formulations for inhaled/intranasal administration of the compound of formula I (or a pharmaceutically acceptable salt, solvate or prodrug thereof) may be formulated to be immediate and/or modified release. Modified release formulations include delayed-, sustained-, pulsed-, controlled dual-, targeted- and programmed-release formulations. Sustained or controlled-release can be obtained by using, for example, poly(D,L-lactic-co-glycolic acid).

### Administration of Compounds of the Invention

In some preferred embodiments, the compounds of the invention are administered orally to a patient. However, the compounds may be administered by any route, including by rectal, pulmonary, sublingual, and parenteral administration. Parenteral administration includes, for example, intravenous, intramuscular, intraarterial, intraperitoneal, intranasal, intravaginal, intravesical (e.g., to the bladder), intradermal, transdermal, topical or subcutaneous administration.

The dosing interval may be once a week, twice a week, every-other-day, once per day, typically once, twice, three times or four times per day with the doses given at equal intervals throughout the day and night in order to maintain a constant presence of the drug. However, those skilled in the art will be aware that a treatment schedule can be optimized for any given subject, and that administration of the compound of formula I (or a pharmaceutically acceptable salt, solvate or prodrug thereof) may occur less frequently than once per day. The treatment may be carried out for as long a period as necessary.

The specific dose of a compound of formula I (or a pharmaceutically acceptable salt, solvate or prodrug thereof) required to elicit a therapeutically beneficial response in the subject will, of course, be determined by the particular circumstances of the individual subject including the size, weight, age and sex of the subject, and the route of administration of the compound.

For example, a daily dosage from about 20-500 mg/day, 50-150 mg/day, 50-200 mg/day, 50-250 mg/day, 250-500 mg/day, or 250 mg-1000 mg/day may be utilized. However, higher or lower doses are also contemplated as it may be necessary to use dosages outside these ranges in some cases. The daily dosage may be divided, such as being divided equally into two to four times per day daily dosing. Moreover, for long-term administration, as might be required in a method of inducing increasing and/or improving healthspan, lifespan, and/or mental acuity of a subject, the compound may, preferably, be administered at a dose of approximately 50 mg/day, 75 mg/day, 100 mg/day, 150 mg/day, 200 mg/day 250 mg/day, 300 mg/day, 350 mg/day, 400 mg/day, 450 mg/day, 500 mg/day, 550 mg/day, 600 mg/day, 650 mg/day, 700 mg/day, 750 mg/day, 800 mg/day, 850 mg/day, 900 mg/day, 950 mg/day, 1000 mg/day, 1050 mg/day, 1100 mg/day, 1150 mg/day, 1200 mg/day, 1250 mg/day, 1300 mg/day, 1350 mg/day, or between 50-150 mg/day, 50-200 mg/day, 50-250 mg/day, 250-500 mg/day, 250 mg-1000 mg/day, or 1000 mg-1500 mg/day or less than 1000 mg/day, or approximately 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, and/or between 4-12 mg/kg/day.

The invention also provides a pharmaceutical pack or kit comprising one or more containers filled with one or more of the ingredients of the pharmaceutical composition of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

Without further description, it is believed that those skilled in the art can, using the preceding description and the following illustrative examples, readily make and utilize the compounds of formula I (for example, the compounds shown in FIG. 1) or a pharmaceutically acceptable salt, solvate or prodrug thereof, and practice methods of the invention. The following examples points out some of the preferred embodiments of the present invention, but is not to be construed as limiting the disclosure in any way. Further, although the invention herein has been described with reference to embodiments, it is to be understood that such embodiments, and examples provided herein, are merely illustrative of the principles and applications of the present invention. It is therefore to be understood that numerous modifications can be made to the illustrative embodiments and examples, and that other arrangements can be devised without departing from the spirit and scope of the present invention as defined by, for example, the claims hereinafter. All patent applications, patents, literature and references cited herein are hereby incorporated by reference in their entirety.

### EXAMPLES

The invention will now be further illustrated with reference to the following examples. It will be appreciated that what follows is by way of example only and that modifications to detail may be made while still falling within the scope of the invention.

### Example 1: Synthesis of Compounds and Metabolism Assays

Polydeuterated compounds 2, 4 and 5 (as shown in FIG. 1) may be synthesized by adapting one or more synthetic scheme described in Latli B et al., J Label Compd Radiopharm 53:15-23, 2010. For example, compound 2 may be synthesized by modifying “Scheme 4” of Latli et al., 2010 by substituting 2-amino-3,5-dibromo-benzaldehyde with 2-amino-3,5-dibromo-4,6-[²H₂]-benzaldehyde (i.e., with two deuteriums replacing the hydrogens on the benzene ring), and reacting the compound as described with polydeuterated 4-aminocyclohexanol. The dideutero-2-amino-3,5-dibromobenzaldehyde can be obtained by methods known to those skilled in the art from several starting materials, including but not limited to the method presented in “Scheme 2” of Latli et al., 2010, starting from a perdeuterated anthranilic acid analog of the ¹⁴C-labeled anthranilic acid used in the first step.

### Example 1.1—Synthesis of Bis-Deuterated Ambroxol a (ZW-001) HCl Salt

The HCl salt of compound bis-deuterated ambroxol A (ZYW-001 used herein but labeled ZW-01 in Scheme 1) was prepared according to Scheme 1.

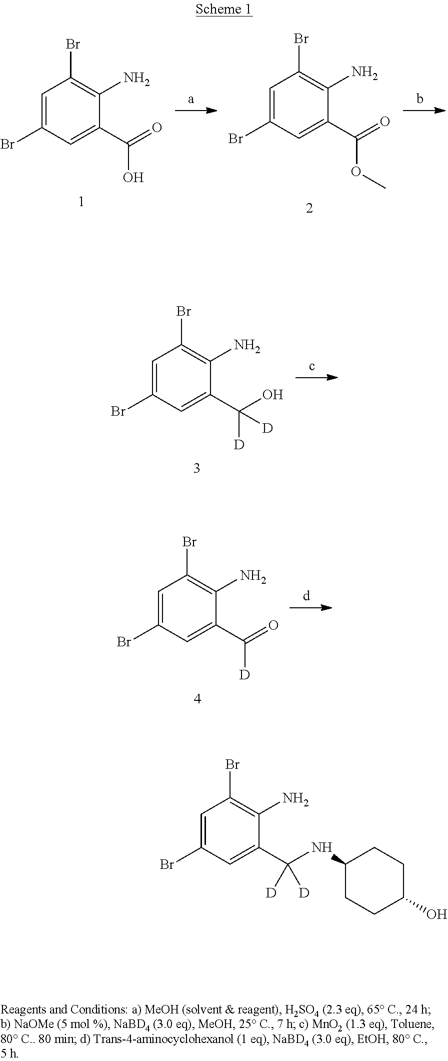 [SMILES: Nc1c(Br)cc(Br)cc1C(=O)O ; COC(=O)c1cc(Br)cc(Br)c1N ; [2H]C([2H])(O)c1cc(Br)cc(Br)c1N ; [2H]C(=O)c1cc(Br)cc(Br)c1N ; [2H]C([2H])(N[C@H]1CC[C@H](O)CC1)c1cc(Br)cc(Br)c1N]

Preparation of Intermediate 2: To a solution of 2-amino-3,5-dibromobenzoic acid 1 (7.0 g, 23.73 mmol) in MeOH (120 ml) was added conc. H₂SO₄ (3 ml, 54.00 mmol). The reaction mixture was refluxed for 24 h (completion of reaction monitored by TLC). After allotted time, reaction mixture was cooled to 0° C. and saturated sodium hydroxide solution was added until pH 5.5 (measured with pH paper) followed by addition of sat′d NaHCO₃ (aq.) until pH 7.5 was reached and white solid precipitated from the reaction mixture. The mixture was filtered, and the residue was washed with ice cold MeOH (25 mL). The filtrate was extracted with 2×50 ml portions of dichloromethane. Combined organic extracts were dried (Na₂SO₄, 75 g), filtered and concentrated to give an off-white, crystalline solid 2 which was used without further purification (3.3 g, 66%). MS (ESI): m/z 309 (M+H)⁺.

Preparation of Intermediate 3: A 50 ml two-necked round-bottom flask, equipped with a magnetic stir bar and fitted with a rubber septum, was charged with 2 (1.5 g, 5 mmol), methanol (10 ml), and 5 mol % of NaOMe (13.5 mg, 0.25 mmol) at 25° C. Reaction was stirred under nitrogen for 20 min and powdered NaBD₄ (627 mg, 15 mmol) was added in one portion with constant stirring. The mixture was stirred under nitrogen for additional 7 h at 25° C. The progress of the reaction was followed by TLC. The reaction was quenched by the addition of 10 ml methanol. The resultant solution was evaporated on a rotary evaporator at reduced pressure, and the residue was resuspended in 30 ml of dichloromethane and filtered through celite. Filtrate was dried over anhydrous sodium sulfate (50 g), and the solvent was evaporated on a rotovap to obtain the crude reaction product 4 (0.75 g, 53%) that was used in subsequent reactions without purification. MS (ESI): m/z 284 (M+H)⁺.

Preparation of Intermediate 4: A solution of crude 3 (0.75 g, 2.65 mmol) in toluene (30 ml) was treated with manganese (IV) oxide (1.63 g, 18.5 mmol, 88%) and the reaction mixture was stirred at 80° C. for 80 minutes. Upon cooling to ambient temperature mixture was filtered through celite. Celite pad was thoroughly washed with toluene (100 ml) and the filtrate concentrated to produce pink solid (0.48 g, 64%) 4 which was used in the subsequent reaction without purification. MS (ESI): m/z 281 (M+H)⁺.

Preparation of ZW-001 HCl Salt: A mixture of 4 (0.48 g, 1.7 mmol) and trans-4-aminocyclohexanol (0.2 g, 1.7 mmol) in ethanol (15 ml) was heated at 80° C. for 5 hours and allowed to cool to ambient temperature. Sodium borodeuteride (0.22 g, 5.1 mmol) was added and stirring at ambient temperature was continued for 2 hours before quenching with sat′d NH₄Cl (aq.) (5 ml). After evacuation of the solvent, solid was resuspended in dichloromethane and filtered over celite. The celite pad was rinsed with 100 ml of dichloromethane and the filtrate was concentrated under reduced pressure resulting in off white solid. Elution through a flash column (silica gel 60, 230-400 mesh, 7% MeOH in EtOAc) gave an off-white crystalline solid which was converted to the HCl salt (90 mg, 12%). MS (ESI) m/z: 380.9 (M+H)⁺. Chemical Purity (LCMS): >99%. 1H NMR (d₄-methanol) δ (ppm): 1.35 (m, 2H), 1.5 (m, 2H), 2.05 (m, 2H), 2.25 (m, 2H), 3.2 (m, 1H), 3.55 (m, 1H), 7.45 (s, 1H), 7.65 (s, 1H).

### Example 1.2—Synthesis of Compound ZW-002

Compound ZW-002 (otherwise referred to as Compound 1 herein and labeled as ZW-02 in Scheme 2) was prepared according to Scheme 2.

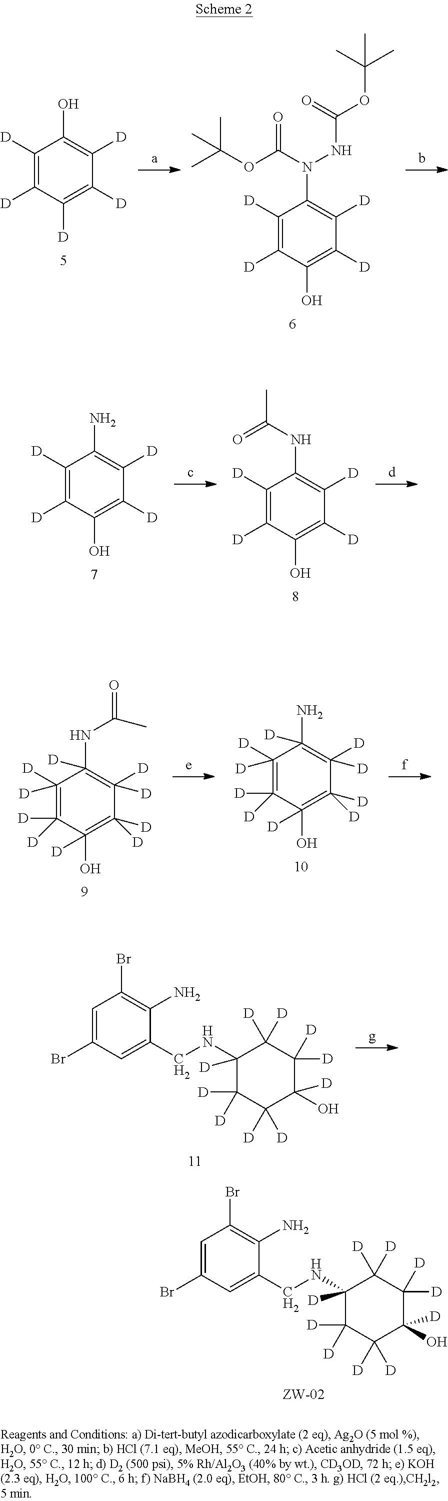

Preparation of Intermediate 6: A mixture of phenol-d₅ (5, 5.0 g, 49.9 mmol) and di-tert-butyl azodicarboxylate (30.0 g, 100 mmol) were added into a 250 ml round bottom flask containing a stirring bar and sealed with a Teflon-lined cap. Then water (150 ml) was introduced. The resulting mixture was stirred form 15 min and then cooled to 0° C. at which point Ag₂O (0.58 g, 5 mol %) was added and the resulting mixture stirred vigorously for 30 minutes. Upon allotted time, the reaction mixture was added to 500 ml of water and extracted with 4×100 ml portions of ethyl acetate. Organic portions were combined and dried over Na₂SO₄ (300 g) then filtered. Subsequently solvent was removed in vacuo resulting in crude yellow solid 6 (13 g, 80%) which was used without further purification. MS (ESI): m/z 217 (fragment)⁺.

Preparation of Intermediate 7: Intermediate 6 (10.0 g, 30 mmol) was added to 150 ml methanol and stirred for 30 min. After allotted time 54 ml of 4N HCl in dioxane (215 mmol) was added and the reaction mixture stirred at 55° C. for 24 h resulting in formation of suspended solids. The mixture was cooled to room temperature and filtered. The residue was washed with 200 ml of ethyl acetate. Resulting light purple solid (2.5 g, 74%), 7 was dried in the vacuum oven at 35° C. for 16 h and used in the next step without further purification. MS (ESI): m/z 114 (M+H)⁺.

Preparation of Intermediate 8: A solution of 7 (2.5 g, 22 mmol) in water (90 ml) was treated with 3.25 ml (34 mmol) of acetic anhydride and the mixture was heated to 55° C. with stirring for 30 min. then cooled to room and stirred for 12 h. After allotted time the mixture was extracted with ethyl acetate (3×100 ml). Organic fractions were combined, washed with brine (50 ml), dried over Na₂SO₄ (200 g), filtered and concentrated in vacuo to give 1.3 g of brown solid (38%), 8 which was used in subsequent steps without purification. MS (ESI) m/z: 156.1 (M+H)⁺.

Preparation of Intermediate 9: A mixture of 8 (1.3 g, 8.4 mmol) and 5% Rh/Al₂O₃ (0.6 g) in deuterated methanol (15 ml) was stirred under 500 psi of deuterium gas in a high-pressure reactor for 72 h. The mixture was filtered through a short pad of Celite, rinsed with methanol and concentrated in vacuo to give 1.43 g of a semi-solid which was used in the next step without purification. MS (ESI) m/z: 168.1 (M+H)⁺.

Preparation of Intermediate 10: A solution of 9 (1.43 g, 8.5 mmol) in aqueous KOH (10 ml, 2N) was heated to a gentle reflux for 6 h. Progress of the reaction was monitored by MS (ESI). After completion, reaction mixture was cooled to room temperature and saturated with NaCl (3.5 g) and then extracted with 3×20 ml portions of CH₃Cl:IPA (3:1), dried over Na₂SO₄ (150 g), and concentrated in vacuo to give 0.3 g (28%) of viscous semisolid 10 which was used in the next step without further purification.

Preparation of Intermediate 11: A mixture of crude 4 (0.5 g, 1.8 mmol) and 10 (0.25 g, 2.2 mmol) in ethanol (25 ml) was heated at 80° C. for 3 hours and allowed to cool to ambient temperature. Sodium borohydride (0.17 g, 4.5 mmol) was added and stirring at ambient temperature was continued for 2 hours before quenching with sat′d NH₄Cl (aq.) (5 ml). After evacuation of the solvent, solid was resuspended in dichloromethane and filtered over celite. The celite pad was rinsed with 100 ml of dichloromethane and the filtrate was concentrated under reduced pressure to give off-white solid (480 mg, 70%), 11 as a mixture of cis/trans isomers. Solid was subjected to SFC conditions to separate isomers. MS (ESI): m/z 389 (M+H)⁺.

Preparation of ZW-002 HCl Salt: A solution of ZW-002 free base (145 mg, 0.4 mmol) in dicholoromethane (5 ml) was treated with 4N HCl in dioxane (0.2 ml, 0.8 mmol). Solution was stirred for 5 min at room temperature and then hexanes were added (15 ml) and resulting solution kept at 2° C. for 24 h resulting in crystallization of ZW-002 HCl Salt. After filtration a white solid (100 mg, 60%) was collected and dried in vacuo at 35° C. overnight. MS (ESI) m/z: 388.9 (M+H)⁺. Chemical Purity (LCMS): >99%. 1H NMR (d₄-methanol) δ (ppm): 4.25 (s, 2H), 7.45 (s, 1H), 7.65 (s, 1H).

### Example 1.3—Synthesis of Compound ZW-003

Compound ZW-003 (otherwise referred to as Compound 3 herein and labeled as ZW-03 in Scheme 3) was prepared according to Scheme 3.

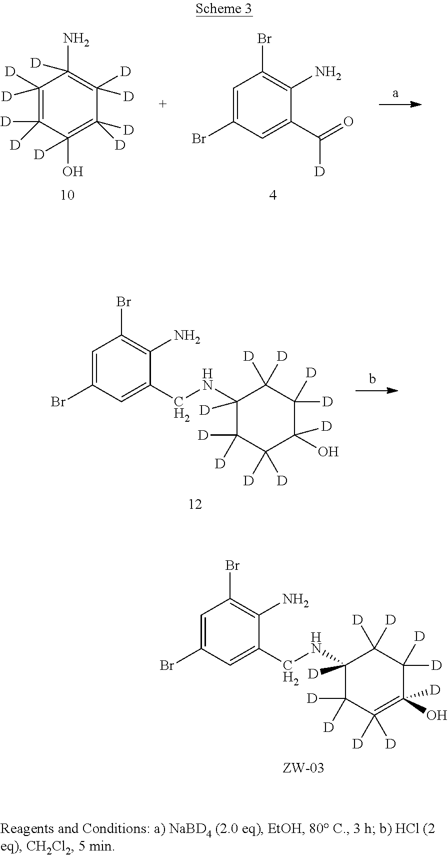

Preparation of Intermediate 12: A mixture of crude 4 (0.5 g, 1.8 mmol) and 10 (0.27 g, 2.2 mmol) in ethanol (25 ml) was heated at 80° C. for 3 hours and allowed to cool to ambient temperature. Sodium borodeuteride (0.23 g, 5.4 mmol) was added and stirring at ambient temperature continued for 2 hours before quenching with sat′d NH₄Cl (aq.) (5 ml). After evacuation of the solvent, solid was resuspended in dichloromethane and filtered over celite. The celite pad was rinsed with 100 ml of dichloromethane and the filtrate was concentrated under reduced pressure to give off-white solid (600 mg, 87%), 12 as a mixture of cis/trans isomers. Solid was subjected to SFC conditions to separate isomers. MS (ESI): m/z 391 (M+H)⁺.

Preparation of ZW-003 HCl Salt: A solution of ZW-003 free base (82 mg, 0.2 mmol) in dicholoromethane (5 ml) was treated with 4N HCl in dioxane (0.1 ml, 0.4 mmol). Solution was stirred for 5 min at room temperature and then hexanes were added (15 ml) and resulting solution kept at 2° C. for 24 h resulting in crystallization of ZW-002 HCl Salt. After filtration off-white solid (60 mg, 70%) was collected and dried in vacuo at 35° C. overnight. MS (ESI) m/z: 391.0 (M+H)⁺. Chemical Purity (LCMS): >99%. 1H NMR (d₄-methanol) δ (ppm): 7.45 (s, 1H), 7.65 (s, 1H).

The stability of the polydeuterated compounds may be readily tested by conducting routine in vitro metabolism assays (e.g., using microsomal incubation to achieve CYP3A4-dependent metabolism); following the disappearance of the assayed compound (e.g., compound 2) and formation of expected metabolite compounds (e.g., carboxylic acid and/or dibromoanthranilic acid). The in vitro metabolism assays of the polydeuterated compounds may be conducted in comparison with, for example, ambroxol and/or other suitable comparator compound(s). It is anticipated that the polydeuteration of the compounds will provide resistance to the metabolism in such assays, as indicated by, for example, slower disappearance of the assayed compound and/or slower formation of the metabolite.

### Example 2: Metabolic Stability of Ambroxol and Analogs in Human Liver Microsomes

The metabolic stability of ambroxol and deuterated analogs was studied in the presence of human liver microsomes to assess the effect of deuteration at different sites on the metabolic stability of ambroxol.

Study design—Test compounds (ambroxol and deuterated analogs) were dissolved in dimethylsulfoxide (DMSO) to prepare 1 mM stock solutions. The compounds were added to preparations of human liver microsomes (200 μL per incubation at a 1 mg/mL protein concentration) such that the final compound concentration was 1 μM. The samples were incubated at 37° C. in the presence of a reduced nicotinamide adenine dinucleotide phosphate (NADPH)-regenerating system (NADP, 1 mM, pH 7.4; glucose-6-phosphate, 5 mM, pH 7.4; glucose-6-phosphate dehydrogenase, 1 unit/mL)). A positive control (midazolam, 1 μM, 0.1 mg/mL protein) was incubated concurrently to ensure the microsome preparation was performing as expected. Samples were obtained at t=0, 30, 60, and 120 min and quenched by addition of acetonitrile.

Quenched samples were analyzed by liquid chromatography coupled to tandem mass spectrometry detection (LC/MS-MS). The concentration of the test compounds at each time point were converted to percent remaining relative to the concentration of the compound at time t=0 (which serves as the 100% value). All data points were included in data processing. The rate constant of elimination (kₑₗ, min⁻¹) of each test compound was determined from the time course of disappearance of the parent compound based on fitting the experimental data to a single exponential decay formula (Aₜ=Aₒe^{-kelt}, where Aₜ is the % remaining at time t, Aₒ is 100%, kₑₗ is the elimination rate constant, and t is time). The in vitro half-life (t_{1/2}) was determined from kₑₗ based on the formula: t_{1/2}=ln(2)/kₑₗ. The rate constant of elimination was also used to calculate the in vitro intrinsic clearance CL_{int-microsomes} (μL/min/mg protein).

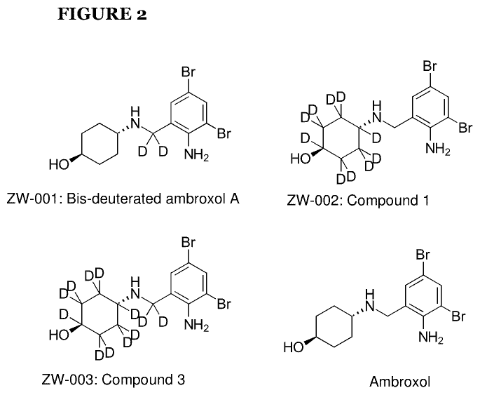

Results-Compound structures are shown in FIG. 2. Estimated in vitro elimination half-lives (t_{1/2}) and in vitro intrinsic clearances (CL_{int-microsomes}) for deuterated compounds ZW-001, ZW-002, and ZW-003 are presented in Table 2 where they are compared to the values obtained for parent compound ambroxol.

TABLE 2
In vitro elimination half-lives (t_{1/2}) and intrinsic
clearances (CL_{int-microsomes}) for compounds studied

| | t_{1/2} | CL_{int-microsomes} |
|---|---|---|
| | (min) | (μL/min/mg protein) |
| ZW-001 | 99 | 7.00 |
| ZW-002 | 90 | 7.70 |
| ZW-003 | 90 | 7.70 |
| ambroxol | 60.8 | 11.4 |

As shown in Table 2, deuteration of ambroxol leads to improved stability against metabolism as the elimination half-life of the deuterated compounds is increased by about 50% vs. ambroxol, irrespective of the deuteration scheme. Interestingly, deuteration of the cyclohexyl ring without deuteration of the methylene group of the aminodibromobenzyl moiety (compound ZW-002) leads to the same improvement in stability as deuteration of the methylene group (compound ZW-001). By contrast, deuteration of both the methylene group and the cyclohexyl ring (compound ZW-003) does not lead to a synergistic or additive stabilization effect as the stability of this compound is characterized by the same elimination half-life.

### Example 3: Prophetic Study with Compound 2 Showing Activity in Improving Lifespan

Compound 2 is a polydeuterated analog of ambroxol. This compound, as a representative example of the compounds of the invention, can be investigated for activity in improving lifespan as follows.

Weaned male BDF1 mice raised from pups, housed 1-4 animals per cage and fed Teklad 7013 NIH-31 rodent chow and water ad libitum, at 2 months of age, are divided into two roughly equal groups of 22-25 animals. One group continues to be fed the Teklad chow as before (control); the other group is switched to Teklad chow formulated with compound 2 at 300 mg per kg of chow. This formulation was designed, based on the average ad libitum chow consumption of an adult male BDF1 mouse, to deliver 50 mg/kg/day (based on body mass) to each mouse in the treated group. Mice are maintained on this diet until they die naturally or until they reach 16 months of age, at which time the experiment ends. During the course of the experiment, all mice are periodically removed from their cages and handled in the course of subjecting them to various sensorimotor, cognitive, and/or behavioral tests.

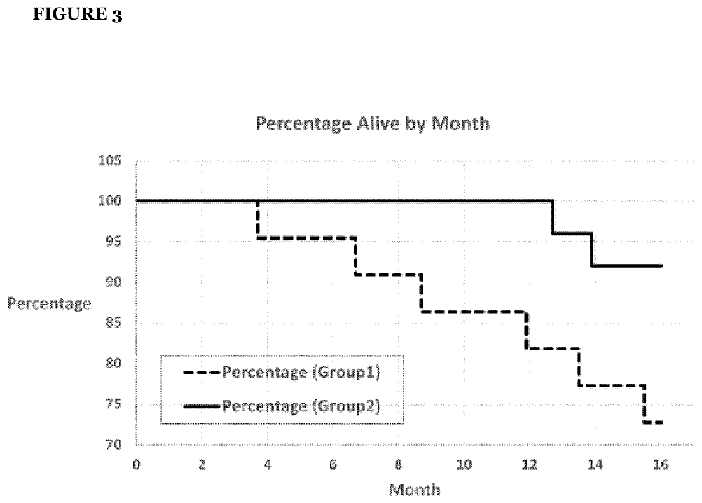

FIG. 3 provides animal data for an experiment as described in the preceding paragraph using ambroxol instead of compound 2. It shows that ambroxol is able to increase lifespan in a mouse animal model. Group 1 represents control animals that were not administered ambroxol; Group 2 represents animals administered 50 mg/kg of ambroxol daily as a chow supplement, starting at 2 months of age. The data also suggests that not only did ambroxol increase lifespan, but that when sacrificed, surviving animals of Group 2 appeared to be at least as healthy, on average, as did the Group 1 animals, indicating that healthspan was extended in parallel with lifespan. It is anticipated that compound 2 will achieve similar results.

### Example 4: Prophetic Study with Compound 2 Showing Activity in Improving Tremor in Mouse Model of PD

Compound 2 can be investigated for activity in improving tremor in a mouse animal model of Parkinson's Disease as follows.

6-OHDA mice (i.e., mice that have been injected with 6-hydroxydopamine into the striatum) showing rest tremor are selected. Three groups of mice, with at least 10 mice in each group, are administered with a “High Dose” (Group 1:150 mg/kg/day of compound 2), “Low Dose” (Group 2:50 mg/kg/day of compound 2), or no dosage of compound 2 (Control Group); and tremor in the mice monitored daily using electromyography or force plate-based measurement (Bekar L et al., Nat Med 14:75-80, 2008) to assess the effect of compound 2 on rest tremor.

### Example 5: Prophetic Study with Compound 2 Showing Effect on Aggregated Aβ in Mouse Model of AD

Compound 2 can be investigated for activity on aggregated deposits of amyloid β peptide (Aβ) in a mouse animal model of Alzheimer's Disease as follows.

An AD mouse model showing significantly elevated production of Aβ is selected (e.g., a mouse including the “Swedish mutation” in the human amyloid precursor protein (APP)). Using three groups of the mice; that is, a “High Dose” Group (150 mg/kg/day of compound 2), a “Low Dose” Group (50 mg/kg/day of compound 2), and a Control Group (no compound 2), analysis can be made after appropriate periods of treatment for the relative development of diffuse and fibrillar deposits of aggregated Aβ by, for example, comparing silver staining or Aβ immunohistochemistry against Congo red or thioflavin-S histology (Jankowsky J et al., Mol Neurodegen 12:89, 2017).

### Example 6: Prophetic Study with Compound 2 Showing Activity in Improving Cognitive Function

Compound 2 can be investigated for activity in improving cognitive function in a mouse animal model as follows.

Three groups of mice, with at least 13 mice in each group, are given a cognitive acuity test at 7 months. The three groups of mice are: “High Dose” Group (150 mg/kg/day of compound 2), “Low Dose” (50 mg/kg/day of compound 2) Group, and a Control Group (no compound 2).

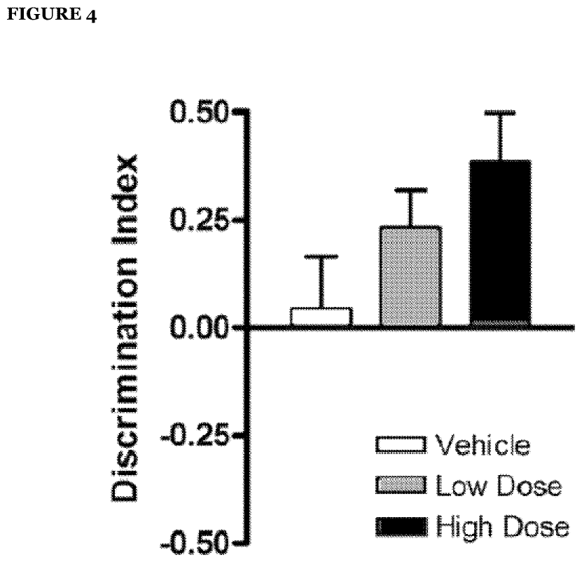

The results of such an experiment, but using ambroxol instead of compound 2, are shown in FIG. 4. A dose-related improvement in cognitive acuity was observed, with the “High Dose” Group showing the best cognitive acuity results, the “Low Dose” Group showing results in between the Control Group and the “High Dose” Group of animals, and the Control Group showing the base line cognitive acuity results. The dosage results are substantially lower than doses previously observed to effectively promote GCase chaperoning activity in mice (e.g., Migdalska-Richards et al., Ann Neurol 80:766-775, 2016).

### Example 7: Prophetic Study with Compound 2 Showing Induction of Macroautophagy

Compound 2 can be investigated for its ability to induce macroautophagy in mouse cells in culture as follows.

Mouse fibroblasts (NIH3T3) obtained from the American Type Culture Collection (ATCC). Cells are maintained in Dulbecco's modified Eagle's medium (DMEM) (Sigma, St. Louis, MO) in the presence of 10% newborn calf serum (NCS), 50 μg/ml penicillin, and 50 μg/ml streptomycin at 37° C. with 5% CO2. Cells plated in glass-bottom 96-well plates are treated for the indicated time and after fixation, images are acquired using a high-content microscope (Operetta, PerkinElmer). For example, images of 9 different fields per well may be captured, resulting in an average of 2,500-3,000 cells. Nuclei and puncta are identified using the manufacturer's software. The number of particles/puncta per cell can be quantified using the “particle identifier” function in the cytosolic region after thresholding in non-saturated images. In all cases, focal plane thickness is set at 0.17 μm and sections with maximal nucleus diameter selected for quantification. Values may be presented as number of puncta per cell section that in acquisition conditions represents 10-20% of the total puncta per cell. Macroautophagy activity in intact cells is measured upon transduction with lentivirus carrying the mCherry-GFP-LC3 tandem construct (Kimura S et al., Autophagy 3 (5): 452-460, 2007). Cells plated on glass-bottom 96 well plates and fluorescence are read in both channels. Puncta positive for both fluorophores correspond to autophagosomes, whereas those positive for only the red fluorophore correspond to autolysosomes. Autophagic flux is determined as the conversion of autophagosomes (yellow) to autolysosomes (red only puncta).

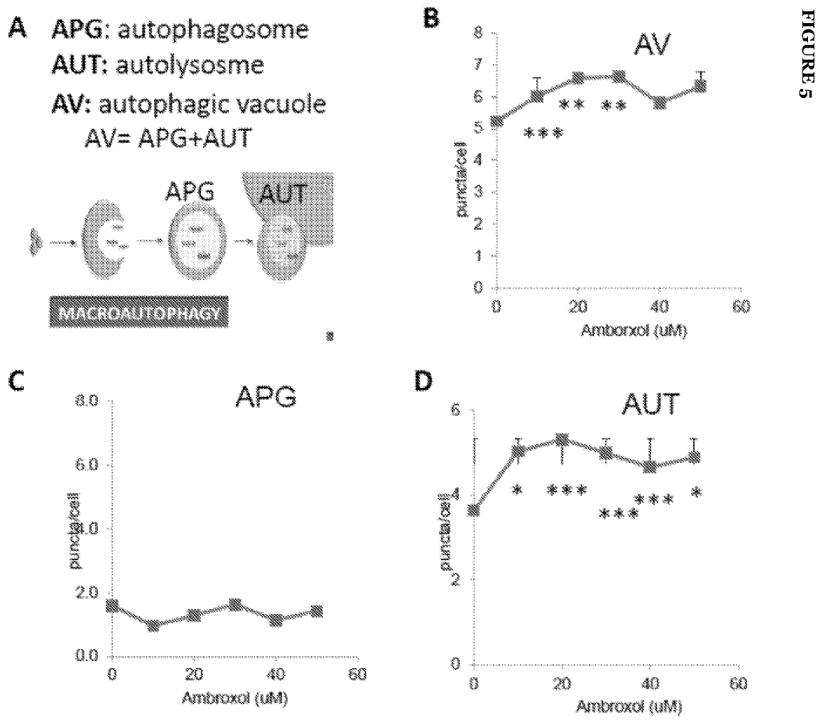

FIG. 5 shows the results of an equivalent experiment using ambroxol instead of compound 2. It appears that ambroxol may, apart from its pharmacological activity as a GCase chaperone, be inhibiting nutrient sensing. By interfering with nutrient sensing, ambroxol may be triggering a response by the cells of the animal appropriate to the organism entering a nutrient-limited or fasting state, thus sending the organism into a “catabolic signaling” mode characterized by lysosome biogenesis, and autophagy induction, leading to improved lifespan, healthspan and/or cognitive acuity (see, e.g., Efeyan et al., Nature 517:302-310, 2015) (hereby incorporated by reference in its entirety). Thus, ambroxol and polydeuterated analogs of ambroxol and related compounds may systemically inhibit nutrient sensing resulting in the whole organism entering catabolic signaling mode.

The human equivalent doses (HEDs), calculated from the “low” and “high” mouse doses of this study, are approximately 4 mg/kg/day and 12 mg/kg/day, which is approximately 250 mg/day or 750 mg/day for an average 62.5 kg human. Thus, in preferred embodiments, long-term administration of a compound of formula I (or a pharmaceutically acceptable salt, solvate or prodrug thereof) administered at a dose of approximately 50 mg/day, 75 mg/day, 100 mg/day, 150 mg/day, 200 mg/day 250 mg/day, 300 mg/day, 350 mg/day, 400 mg/day, 450 mg/day, 500 mg/day, 550 mg/day, 600 mg/day, 650 mg/day, 700 mg/day, 750 mg/day, 800 mg/day, 850 mg/day, 900 mg/day, 950 mg/day, 1000 mg/day, 1050 mg/day, 1100 mg/day, 1150 mg/day, 1200 mg/day, or between 50-150 mg/day, 50-200 mg/day, 50-250 mg/day, 250-500 mg/day, or 250 mg-1000 mg/day, or less than 1000 mg/day, or approximately 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, and/or between 4-12 mg/kg/day may be expected to be effective in improving healthspan, lifespan, and/or mental acuity.

### Example 8: Formulation of Polydeuterated Analogs into High Drug Loading Liquid Oral Pharmaceutical Compositions and Properties Thereof

Preparation of granules—Suitable granules of compound 2 (as a representative example of the compounds of the invention) can be prepared as follows: Micronized compound 2 in the form of a powder is placed into a rotor granulator (GXR-35 Rotor Granulator, Freund-Vector Corporation) and a binder solution of hydroxypropylcellulose (HPC-Klucel LF) sprayed onto the ambroxol HCl powder to form a granular core. Additional compound 2 powder is then co-sprayed with the binder solution to grow the spheres. As layers of compound 2 are added, the particles became more spherical. Resulting spheres may contain 97% by weight of compound 2 and 3% by weight of HPC. A particle size (x₅₀) of about 350 microns and a density of about 0.7 g/ml may be achieved.

Application of a water-soluble seal coating to the granules—The compound 2 granules may be seal coated to generate a smooth and uniform substrate using a bottom-spray fluidized bed coater equipped with a Wurster column. Batch size may be approximately 750 grams. A suitable seal coating contains 9.1% by weight of hypromellose (HPMC); 0.9% by weight of triethyl citrate (TEC); and 90% by weight of water. The granules can be seal coated to a 2% weight gain.

Application of enteric coating to the granules—Seal coated compound 2 granules may be enteric coated using a bottom-spray fluidized bed coater equipped with a Wurster column. Batch size may be approximately 750 grams. A suitable enteric coating contains 58.0% by weight of Eudragit L30D55; 0.9% by weight of triethyl citrate (TEC); 8.7% by weight of Plasacryl T20; and 32.5% by weight of water. The seal coated granules can be enteric coated to a 35% weight gain.

Dissolution Properties—The enteric coated granules can be tested for enteric dissolution properties. The dissolution parameters of equivalent granules comprising ambroxol hydrochloride are set forth in Table 3.

TABLE 3
Dissolution Parameters

| Apparatus | II (Paddles) | |
|---|---|---|
| Phase I Media | 750 mL 0.1N HCl | |
| (0-120 minutes) | | |
| Phase II Media | Add 250 mL 0.2M sodium phosphate | |
| (120-240 minutes) | tribasic to Phase 1. | |
| | Adjust pH to 6.8 | |
| Paddle Speed | 75 | rpm |
| Sample Timepoints | 120, 135, 150, 165, 180, 240 minutes | |
| Sample Volume | 2 | mL |

| | |
|---|---|
| Number of Samples | N = 2 |

### Example 9: Studies on the Effects of Deuterated Ambroxol on Human iPSC-Derived Neurons

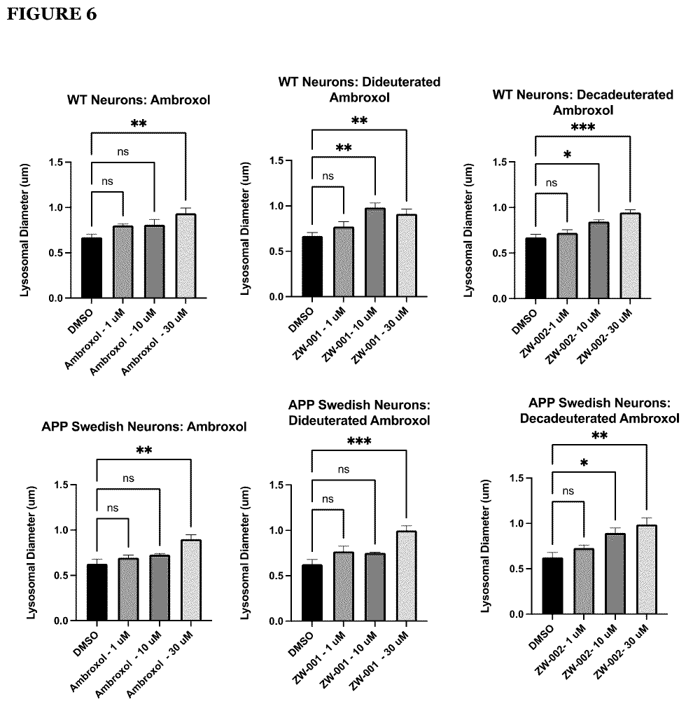

The effects of deuterated ambroxol on human iPSC-derived neurons were investigated and the results are provided in FIG. 6. Human iPSC-derived neurons were grown in culture for 14 days. Lysosomes were stained with ‘Lysotracker’ and cells were fixed and imaged using confocal microscopy. Lysosomal size was measured using IMARIS software (Bitplane).

### Example 10: Studies on the Effects of Deuterated Ambroxol on Lysosomal, Autophagosomal and TFEB Gene Expression

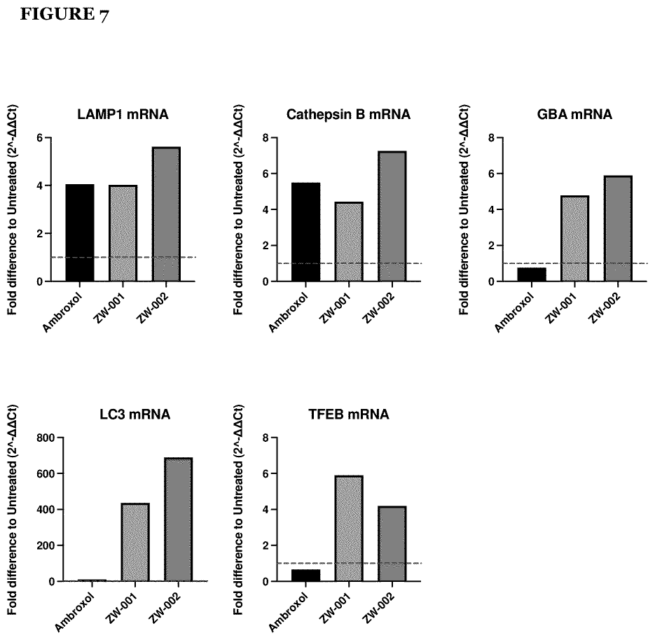

The effects of deuterated ambroxol on lysosomal, autophagosomal and TFEB gene expression were investigated and the results are provided in FIG. 7. Mouse Neuroblastoma (N2A) cells were treated for 3 days with solvent alone (DMSO), ambroxol or deuterated ambroxol Compound D1 or D2 at 10 microMolar. Cells were extracted and mRNAs were quantitated by qPCR.

The data obtained in Examples 9 and 10 show or indicate that:
The cells all grow in the presence of the compounds
All the compounds increase lysosomal size in human neurons, and in human neurons bearing an Alzheimer's disease-causing mutation.
It may be inferred from this that lysosomal genes in general, and not just beta-glucocerebrosidase (UniProt Entry PO4062) (“GBA”) function is improved.
All the compounds increase the amount of lysosomal and autophagosomal gene expression, and TFEB expression.
Some of these increases are substantial improvements over the original ambroxol compound.

The disclosures of each and every patent, patent application, and publication cited herein are hereby incorporated herein by reference in their entirety.

While the invention has been disclosed with reference to specific embodiments, it is apparent that other embodiments and variations of this invention may be devised by those skilled in the art without departing from the true spirit and scope of the invention. The appended claims are intended to be construed to include all such embodiments and equivalent variations.

What is claimed:

1. A polydeuterated compound according to formula I: 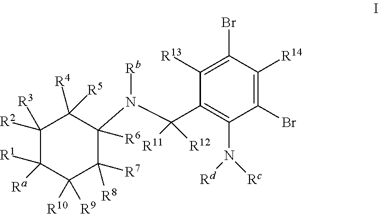
wherein
R^{a} is selected from H, hydroxyl (OH), lower alkyl, and lower alcohol) wherein optionally one or more H atoms in any of the aforementioned groups is replaced by deuterium (D),
R^{b} is selected from deuterium H, (D) and 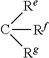
where R^{e}, R^{f} and R^{g} are independently selected from H and D,
R^{c} and R^{d} are independently selected from H and D, and
each of R¹ to R¹⁴ are independently selected from H and D; and
wherein the polydeuterated compound comprises at least two deuterium (D) atoms, and with the proviso that the compound is not bis-deuterated ambroxol A or [D₁₁]-ambroxol B shown below;
or a pharmaceutically acceptable salt, solvate or prodrug thereof:
A: B:

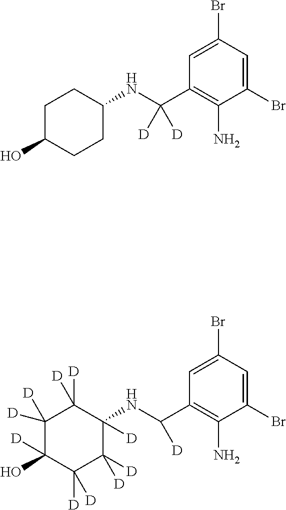 [SMILES: [2H]C([2H])(N[C@H]1CC[C@H](O)CC1)c1cc(Br)cc(Br)c1N ; [2H]C(N[C@]1([2H])C([2H])([2H])C([2H])([2H])[C@@]([2H])(O)C([2H])([2H])C1([2H])[2H])c1cc(Br)cc(Br)c1N]

2. A compound according to claim 1 comprising at least 3 deuterium (D) atoms.

3. A compound according to claim 1 comprising at least 10 deuterium (D) atoms.

4. A compound according to claim 1, wherein the deuterium atoms are provided only on the ring structures of the compound of formula I.

5. A compound according to any one of claims 1 to 4, wherein at least one of R¹¹ and R¹² is deuterium (D).

6. A compound according to claim 5, wherein both of R¹¹ and R¹² are deuterium (D).

7. A compound according to any one of claims 1 to 4, wherein the compound is deuterated: only at each of R¹ to R¹⁰; only at each of R¹ to R¹⁰, R¹¹ and R¹²; only at each of R¹ to R¹⁰, R¹³ and R¹⁴; or only at each of Ru to R¹⁴.

8. A compound according to any one of claims 1 to 4, wherein the compound is deuterated at each of R¹ to R¹⁴.

9. A compound according to any one of claims 1 to 8, wherein the compound is a polydeuterated analog of ambroxol or a polydeuterated analog of bromhexine.

10. A compound according to claim 1, wherein the compound is selected from:

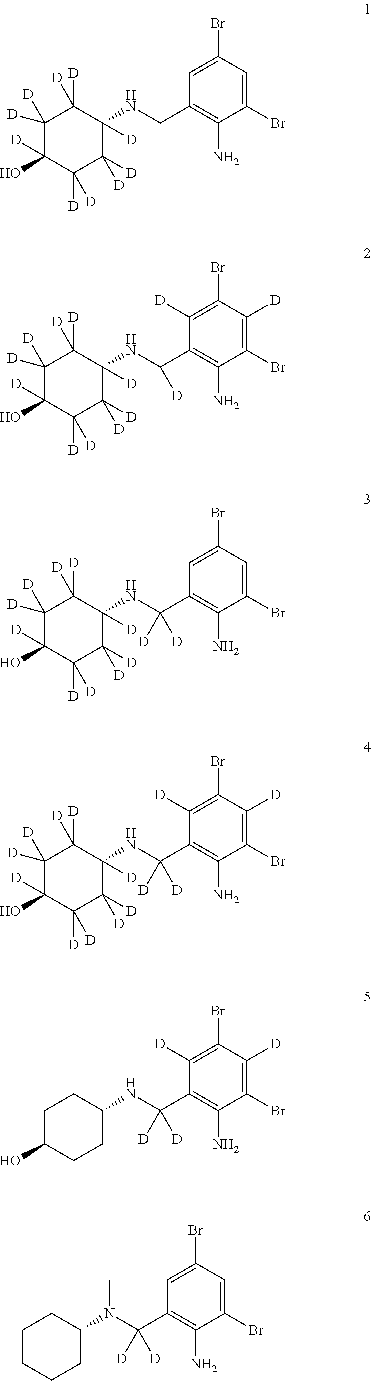

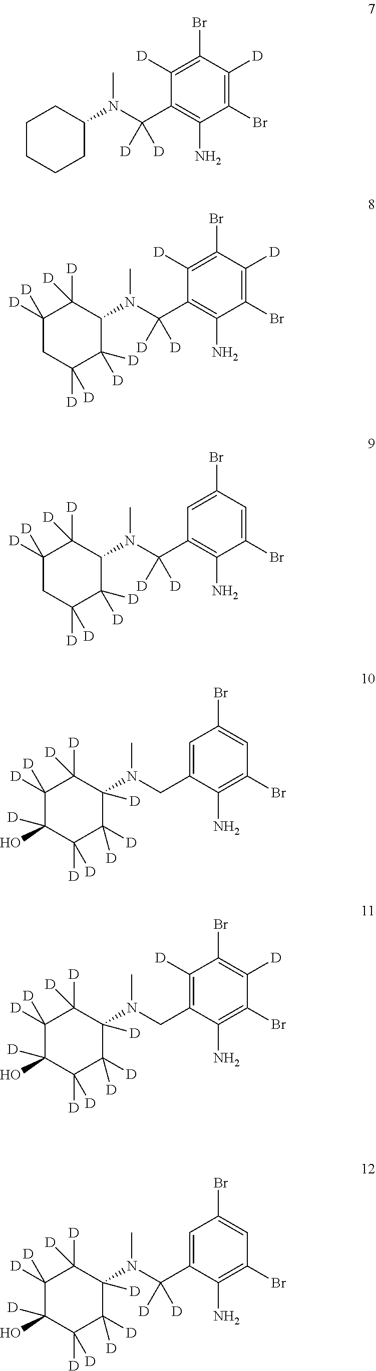

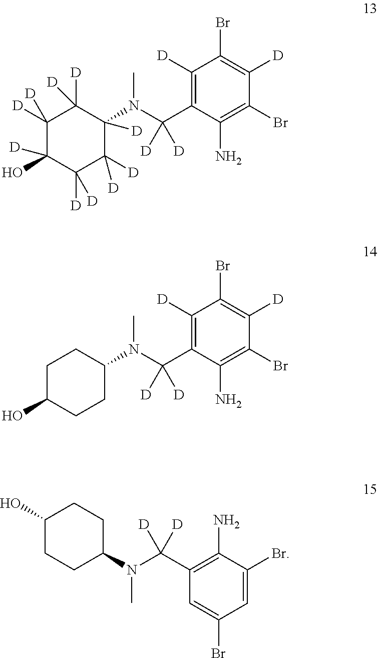 [SMILES: [2H]c1c(Br)c([2H])c(C([2H])([2H])N(C)[C@]2([2H])C([2H])([2H])C([2H])([2H])[C@@]([2H])(O)C([2H])([2H])C2([2H])[2H])c(N)c1Br ; [2H]c1c(Br)c([2H])c(C([2H])([2H])N(C)[C@H]2CC[C@H](O)CC2)c(N)c1Br ; [2H]C([2H])(c1cc(Br)cc(Br)c1N)N(C)[C@H]1CC[C@H](O)CC1]

11. A pharmaceutical composition comprising a compound (or a pharmaceutically acceptable salt, solvate or prodrug thereof) according to any one of claims 1 to 10, optionally in combination with a pharmaceutically acceptable carrier.

12. A composition according to claim 11, wherein the composition is a liquid oral pharmaceutical composition.

13. A composition according to claim 12 comprising a high loading of a compound of formula I (or a pharmaceutically acceptable salt, solvate or prodrug thereof) according to any one of claims 1 to 10, wherein the composition comprises (i) the compound of formula I (or a pharmaceutically acceptable salt, solvate or prodrug thereof); and (ii) at least one pharmaceutically acceptable excipient, and wherein the compound of formula I (or a pharmaceutically acceptable salt, solvate or prodrug thereof) is in the form of granules having a granular core comprising from about 60 to about 97 weight percent of an active pharmaceutical ingredient and from about 3 to about 40 weight percent of the excipient, wherein the weight percent is based on the total weight of the granular core.

14. A composition according to claim 12 comprising a high loading of a compound of formula I (or a pharmaceutically acceptable salt, solvate or prodrug thereof) according to any one of claims 1 to 10, wherein the composition comprises (i) the compound of formula I (or a pharmaceutically acceptable salt, solvate or prodrug thereof); (ii) at least one pharmaceutically acceptable excipient; and (iii) a diluent, wherein the compound of formula I (or a pharmaceutically acceptable salt, solvate or prodrug thereof) is in the form of granules having a granular core comprising from about 60 to about 97 weight percent of an active pharmaceutical ingredient and from about 3 to about 40 weight percent of the excipient, wherein the weight percent is based on the total weight of the granular core; wherein the granule core is coated with (iv) a water-soluble seal coating in an amount to provide from about 0.5 to about 5 percent weight gain, and (v) an enteric coating in an amount to provide from about 0.5 to about 50 percent weight gain.

15. A method of preventing and/or treating a disease or medical condition in a subject selected from the group consisting of respiratory diseases and conditions, lysosomal storage disorders (LSDs), and neurological diseases and conditions, said method comprising administering to the subject an effective amount of a compound (or a pharmaceutically acceptable salt, solvate or prodrug thereof) according to any one of claims 1 to 10 or a pharmaceutical composition according to any one of claims 11 to 14.

16. A method according to claim 15, wherein the disease or medical condition to be prevented and/or treated is a bronchopulmonary disease, or is Gaucher's disease, Pompe disease or Fabry disease, or is Parkinson's disease, dementia with Lewy bodies, Alzheimer's Disease, or Frontotemporal Dementia.

17. A method comprising administering to the subject an effective amount of a compound (or a pharmaceutically acceptable salt, solvate or prodrug thereof) according to any one of claims 1 to 10 or a pharmaceutical composition according to any one of claims 11 to 14, wherein the method is for extending life expectancy of a subject, or for treating, inhibiting or reducing aging of a subject, or for treating, inhibiting or reducing an age-related symptom or an age-related disease in a subject, or for increasing the healthspan, lifespan and/or mental acuity of a subject.

18. A method for preventing and/or treating, reducing symptoms of, and/or slowing the progression of, Alzheimer's disease (AD) or other diseases associated with pathological protein misfolding, aggregation and deposition (including Parkinson's disease (PD), Huntingdon's disease (HD) and Frontotemporal degeneration (FTD)) in a subject, said method comprising administering to the subject an effective amount of ambroxol (or a related compound such as ambroxol hydrochloride and bromhexine) or a compound (or a pharmaceutically acceptable salt, solvate or prodrug thereof) according to any one of claims 1 to 10, in combination with one or more suitable anti-beta amyloid antibody or fragment thereof.

19. A method for preventing, reducing symptoms of, and/or slowing the progression of, Alzheimer's disease (AD) or other diseases associated with pathological protein misfolding, aggregation and deposition (including Parkinson's disease (PD), Huntingdon's disease (HD) and Frontotemporal degeneration (FTD)), comprising administering to the subject an effective amount of ambroxol (or a related compound such as ambroxol hydrochloride and bromhexine) or a compound (or a pharmaceutically acceptable salt, solvate or prodrug thereof) according to any one of claims 1 to 10.

20. A method according to claim 18 or 19, wherein the subject is selected by assaying for a biomarker indicative of an at-risk patient or patient in an early stage of development of AD or other diseases associated with pathological protein misfolding, aggregation and deposition (including Parkinson's disease (PD), Huntingdon's disease (HD) and Frontotemporal degeneration (FTD)).

21. A method according to claim 20, wherein the subject is selected by assaying for a phosphorylated tau protein (p-tau) indicative of a patient at-risk of AD or a patient in an early stage of development of AD.

22. A method according to claim 18 or 19, wherein the subject is selected by genotyping of at least one gene or locus indicative of a patient at-risk of AD or other diseases associated with pathological protein misfolding, aggregation and deposition (including Parkinson's disease (PD), Huntingdon's disease (HD) and Frontotemporal degeneration (FTD)).

23. A method according to claim 22, wherein the subject is selected by genotyping the ApoE gene, p-tau217, p-tau181, p-tau231, p-tau235, and/or N3pG.

24. A method according to claim 23, wherein the subject is selected by genotyping for the ε4 allele of the ApoE gene, p-tau217, p-tau181, p-tau231, p-tau235, and/or N3pG.

25. A method according to any one of claims 18 to 24, wherein the ambroxol (or related compound) or compound (or a pharmaceutically acceptable salt, solvate or prodrug thereof) according to any one of claims 1 to 10, is administered to the subject in a daily dosage selected from a dosage that:
(i) provides a peak concentration in serum of the subject that is greater than 1 μM such as, for example, 2-50 μM, 2-25 μM or 10-20 μM;
(ii) provides a peak concentration in brain tissue of the subject that is greater than 3 μM such as, for example, 5-50 μM, 5-25 μM or 10-20 μM; or
(iii) is in the range of about 250 mg-1000 mg/day or 750-1000 mg/day.
